(19) United States
(12) Patent Application Publication
(10) Pub. No.: US 2023/0091997 A1
(43) Pub. Date: Mar. 23, 2023

(54) BLOOD PRESSURE MEASUREMENT METHOD AND ELECTRONIC DEVICE

(71) Applicant: Samsung Electronics Co., Ltd., Suwon-si (KR)

(72) Inventor: Seongho CHO, Suwon-si (KR)

(21) Appl. No.: 17/994,204

(22) Filed: Nov. 25, 2022

Related U.S. Application Data
(63) Continuation of application No. PCT/KR2021/006040, filed on May 14, 2021.

(30) Foreign Application Priority Data
May 28, 2020 (KR) 10-2020-0064512

(51) Int. Cl. A61B 5/024; A61B 5/11; A61B 5/00

(52) U.S. Cl. CPC A61B 5/02438; A61B 5/02416; A61B 5/1118; A61B 5/1116; A61B 5/4809; A61B 5/4818; A61B 5/742; A61B 5/681

(57) ABSTRACT

An electronic device is provided. The electronic device includes a biometric sensor for detecting biometric data, a situation sensing module, memory, and a processor operatively coupled to the biometric sensor, the situation sensing module, and the memory. The processor can check whether a user is in an inactive state using the situation sensing module, check reference biometric data corresponding to the inactive state if the user is in the inactive state, extract biometric data of the user using the biometric sensor, and measure the blood pressure of the user on the basis of the extracted biometric data and the reference biometric data.

### CROSS-REFERENCE TO RELATED APPLICATION(S)

This application is a continuation application, claiming priority under § 365(c), of an International application No. PCT/KR2021/006040, filed on May 14, 2021, which is based on and claims the benefit of a Korean patent application number 10-2020-0064512, filed on May 28, 2020, in the Korean Intellectual Property Office, the disclosure of which is incorporated by reference herein in its entirety.

### BACKGROUND

### 1. Field

The disclosure relates to a method and an electronic device for measuring blood pressure.

### 2. Description of Related Art

Recently, active research on information technology (IT)-medical convergence technology that combines IT technology and medical technology is being conducted. In particular, monitoring of the health status of a human body is not limited to hospitals, but is expanding to the field of mobile health care that can measure and identify the health status of a user without restriction of time or place in daily life.

Representative types of biosignals include electrocardiography (ECG), photoplethysmography (PPG), and electromyography (EMG) signals, and various biometric sensors for measuring the biosignals in daily life are being developed. In particular, in the case of a PPG sensor, it is possible to analyze the shape of a pulse wave reflecting the cardiovascular state, etc., and measure the blood pressure of the human body.

The above information is presented as background information only to assist with an understanding of the disclosure. No determination has been made, and no assertion is made, as to whether any of the above might be applicable as prior art with regard to the disclosure.

### SUMMARY

In order to continuously measure blood pressure during daily life, a wearable device in which a biometric sensor is mounted may be used. In general, when blood pressure is measured, the wearable device may restrict the posture and activity of a user so that the user measures the blood pressure in a stable state (e.g., a sitting posture or a comfortable posture). For example, the wearable device may guide the user to stop excessive physical activity and measure blood pressure while catching a breath for a predetermined time in a sitting position. The user needs to refrain from certain physical activities to measure blood pressure.

Aspects of the disclosure are to address at least the above-mentioned problems and/or disadvantages and to provide at least the advantages described below. Accordingly, an aspect of the disclosure is to provide a method and an electronic device for automatically measuring blood pressure when a predetermined condition is satisfied during a user's daily life.

Additional aspects will be set forth in part in the description which follows and, in part, will be apparent from the description, or may be learned by practice of the presented embodiments.

In accordance with an aspect of the disclosure, an electronic device is provided. The electronic device includes a biometric sensor configured to detect biometric data, a situation sensing module, a memory, and/or a processor configured to be operatively coupled to the biometric sensor, the situation sensing module, and the memory. The processor may determine identify a user is in an inactive state using the situation sensing module, may identify reference biometric data corresponding to the inactive state when the user is in the inactive state, may extract biometric data of the user using the biometric sensor, and may measure the blood pressure of the user based on the extracted biometric data and the reference biometric data.

In accordance with another aspect of the disclosure, a blood pressure measurement method is provided. The blood pressure measurement method includes identifying whether a user is in an inactive state using a situation sensing module including at least one sensor, identifying reference biometric data corresponding to the inactive state when the user is in the inactive state, extracting biometric data of the user using a biometric sensor, and measuring the blood pressure of the user based on the extracted biometric data and the reference biometric data.

According to various embodiments of the disclosure, an electronic device may detect a user's physical activity (e.g., moving, speaking, and/or sleeping) and may measure and record blood pressure of the user when the user's physical activity satisfies a predetermined condition.

According to various embodiments, the user may automatically measure his/her own blood pressure during daily life, and may identify a change in the blood pressure under a specific condition. According to an embodiment, the user may identify a health state in a specific situation and may take an appropriate action for health. The user may continuously monitor the health state. In addition, various effects directly or indirectly identified through this document may be provided.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a block diagram illustrating an electronic device in a network environment according to an embodiment of the disclosure;

FIG. 2 is a perspective view illustrating an electronic device according to an embodiment of the disclosure;

FIG. 3 is a block diagram illustrating an electronic device according to an embodiment of the disclosure;

FIG. 4A is a diagram illustrating a case in which a first electronic device (e.g., a wearable device) is at least partially controlled by a second electronic device (e.g., a portable electronic device) according to an embodiment of the disclosure;

FIG. 4B is a diagram illustrating data exchange between a first electronic device (e.g., a wearable device) and a second electronic device (e.g., a portable electronic device) according to an embodiment of the disclosure;

FIG. 5 is a flowchart illustrating a method of measuring blood pressure in an electronic device according to an embodiment of the disclosure;

FIG. 6 is a flowchart illustrating a method of measuring blood pressure by detecting a sleeping state by an electronic device according to an embodiment of the disclosure;

FIG. 7 is a flowchart illustrating a method of recording reference biometric data in a calibration operation according to an embodiment of the disclosure;

FIG. 8 is a diagram illustrating a process of acquiring reference biometric data in response to a user's posture according to an embodiment of the disclosure;

FIG. 9A is a first diagram illustrating a user interface displayed in a calibration operation according to an embodiment of the disclosure;

FIG. 9B is a second diagram illustrating a user interface displayed in a calibration operation according to an embodiment of the disclosure;

FIG. 9C is a third diagram illustrating a user interface displayed in a calibration operation according to an embodiment of the disclosure;

FIG. 9D is a fourth diagram illustrating a user interface displayed in a calibration operation according to an embodiment of the disclosure;

FIG. 9E is a fifth diagram illustrating a user interface displayed in a calibration operation according to an embodiment of the disclosure;

FIG. 9F is a sixth diagram illustrating a user interface displayed in a calibration operation according to an embodiment of the disclosure;

FIG. 9G is a seventh diagram illustrating a user interface displayed in a calibration operation according to an embodiment of the disclosure;

FIG. 10 is a diagram illustrating a plurality of pieces of context information that can be detected by an electronic device according to an embodiment of the disclosure;

FIG. 11 is a diagram illustrating a list recorded based on biometric data measured by an electronic device and context information of a user according to an embodiment of the disclosure; and

FIG. 12 is a diagram illustrating a process of measuring blood pressure of a user by an electronic device according to an embodiment of the disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

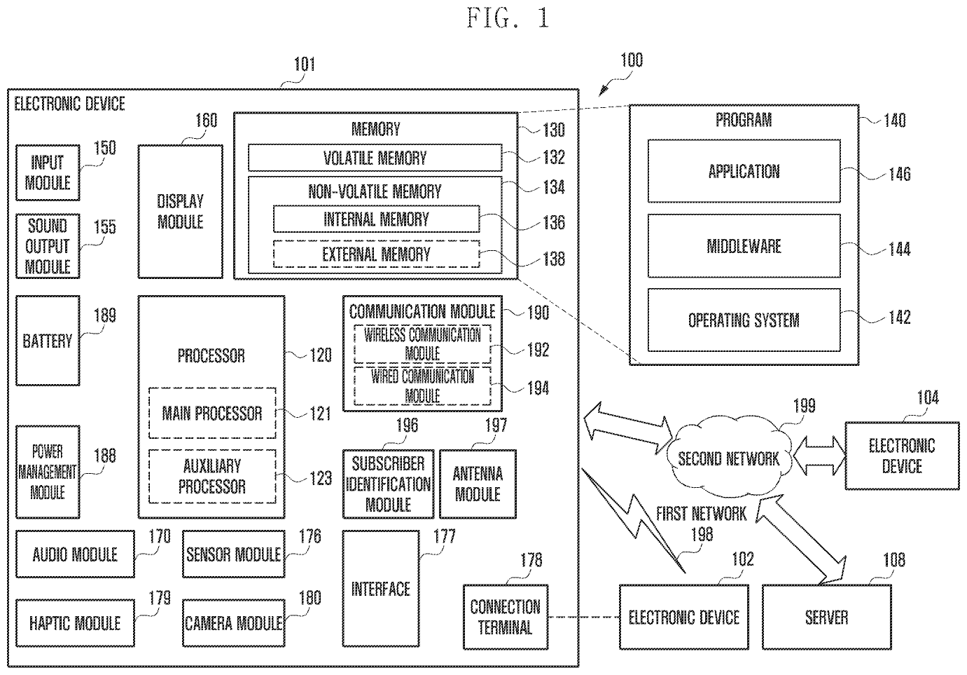

FIG. 1 is a block diagram illustrating an electronic device in a network environment according to an embodiment of the disclosure. According to various embodiments, the electronic device 101 may include a wearable device.

Referring to FIG. 1, the electronic device 101 in the network environment 100 may communicate with an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input device 150, a sound output device 155, a display device 160, an audio module 170, a sensor module 176, an interface 177, a haptic module 179, a camera module 180, a power management module 188, a battery 189, a communication module 190, a subscriber identification module (SIM) 196, or an antenna module 197. In some embodiments, at least one (e.g., the display device 160 or the camera module 180) of the components may be omitted from the electronic device 101, or one or more other components may be added in the electronic device 101. In some embodiments, some of the components may be implemented as single integrated circuitry. For example, the sensor module 176 (e.g., a fingerprint sensor, an iris sensor, or an illuminance sensor) may be implemented as embedded in the display device 160 (e.g., a display).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may load a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), and an auxiliary processor 123 (e.g., a graphics processing unit (GPU), an image signal processor (ISP), a sensor hub processor, or a communication processor (CP)) that is operable independently from, or in conjunction with, the main processor 121. Additionally or alternatively, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display device 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input device 150 may receive a command or data to be used by another component (e.g., the processor 120) of the electronic device 101, from the outside (e.g., a user) of the electronic device 101. The input device 150 may include, for example, a microphone, a mouse, a keyboard, or a digital pen (e.g., a stylus pen). An input device 150 may include a physical key for at least partially controlling the electronic device 101. For example, the input device 150 may include a volume key for controlling the volume level of audio and a key for executing at least one program (e.g., Bixby).

The sound output device 155 may output sound signals to the outside of the electronic device 101. The sound output device 155 may include, for example, a speaker or a receiver. The speaker may be used for general purposes, such as playing multimedia or playing record, and the receiver may be used for an incoming call. According to an embodiment, the receiver may be implemented as separate from, or as part of the speaker.

The display device 160 may visually provide information to the outside (e.g., a user) of the electronic device 101. The display device 160 may include, for example, a display, a hologram device, or a projector and control circuitry to control a corresponding one of the display, hologram device, and projector. According to an embodiment, the display device 160 may include touch circuitry adapted to detect a touch, or sensor circuitry (e.g., a pressure sensor) adapted to measure the intensity of force incurred by the touch.

The audio module 170 may convert a sound into an electrical signal and vice versa. According to an embodiment, the audio module 170 may obtain the sound via the input device 150, or output the sound via the sound output device 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, an HDMI connector, a USB connector, an SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, Wi-Fi direct, or infrared data association (IrDA)) or the second network 199 (e.g., a long-range communication network, such as a cellular network, the Internet, or a computer network (e.g., LAN or wide area network (WAN))). These various types of communication modules may be implemented as a single component (e.g., a single chip), or may be implemented as multi components (e.g., multi chips) separate from each other. According to an embodiment, the wireless communication module 192 may identify and authenticate the electronic device 101 in a communication network, using subscriber information stored in the subscriber identification module 196.

The antenna module 197 may transmit or receive a signal or power to or from the outside (e.g., the external electronic device) of the electronic device 101. The antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. Another component (e.g., a radio frequency integrated circuit (RFIC)) other than the radiating element may be additionally formed as part of the antenna module 197.

According to an embodiment, commands or data may be transmitted or received between the electronic device 101 and the external electronic device 104 via the server 108 coupled with the second network 199. Each of the electronic devices 102 and 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, or client-server computing technology may be used, for example.

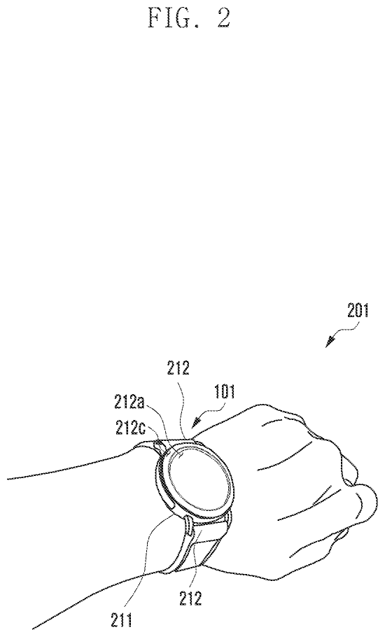

FIG. 2 is a perspective view illustrating an electronic device according to an embodiment of the disclosure.

Referring to FIG. 2 depicting perspective view 201, in various embodiments, the electronic device 101 (e.g., a wearable device) (hereinafter, referred to as a wearable device) is illustrated as an electronic device worn on a user's wrist, but is not limited thereto. The wearable device 101 may include a portable electronic device that is worn while being at least partially in contact with the human body. The wearable device 101 may extract user's biometric data through the skin portion where the blood vessels are located while continuously maintaining a state in which the portable electronic device is at least partially in contact with the human body when the wearable device 101 is worn. In the wearable device 101, a biometric sensor 210 (e.g., a photoplethysmography (PPG) sensor) for extracting user's biometric data may be mounted therein. According to an embodiment, an area in which the biometric sensor 210 is disposed may maintain a state of being at least partially in contact with the human body, and may be provided to correspond to the skin where the user's blood vessels are located.

Referring to FIG. 2, the wearable device 101 may include a housing 211 configured to include a first surface (or front surface) 212a, a second surface (or rear surface) (not shown), and a side surface 212c surrounding a space between the first surface 212a and the second surface, and a binding member 212 (e.g., a strap) configured to be connected to at least a portion of the housing 211 and to be detachably attached to a user's body portion (e.g., wrist, ankle, etc.). According to an embodiment, the first surface 212a may be formed by a front plate (e.g., a glass plate including various coating layers, or a polymer plate) of which at least a portion is substantially transparent. The second surface (not shown) may be formed by a substantially opaque rear plate. The rear plate may be made, for example, by coated or tinted glass, ceramic, polymer, metal (e.g., aluminum, stainless steel (STS), or magnesium), or a combination of at least two of the above materials. The second surface (e.g., the rear surface) of the wearable device 101 may be a surface in direct contact with the human body. The side surface 212c is partially coupled to the front plate (e.g., the first surface 212a) and the rear plate, and may be formed by a side bezel structure (or “side member”) containing a metal and/or a polymer.

In some embodiments, the rear plate and the side bezel structure are integrally formed and may contain the same material (e.g., a metal material such as aluminum). The binding member 212 may be made of various materials and shapes. For example, the binding member 212 may be integrally formed by a woven fabric, leather, rubber, urethane, metal, ceramic, or a combination of at least two of the above materials, or may be formed in such a manner that a plurality of unit links may be formed to be movable with each other. According to an embodiment, the wearable device 101 is not limited to a portable electronic device that is attached to the wrist. The wearable device 101 may include a portable electronic device in which a sensing area of the biometric sensor is at least partially in contact with the human body to measure the user's blood pressure. For example, the sensing area of the biometric sensor may be provided to correspond to the skin where the user's blood vessels are located.

According to various embodiments, the wearable device 101 may include a biometric sensor (not shown) for measuring the user's blood pressure. For example, the biometric sensor may be at least partially disposed on the second surface (e.g., the rear surface) of the wearable device 101 to be at least partially in contact with the human body. The wearable device 101 may utilize a partial area corresponding to a location where the biometric sensor is disposed as a sensing area. The sensing area of the biometric sensor may include an area in direct contact with the human body when the wearable device 101 is worn, and may be provided to correspond to the skin where the user's blood vessels are located. According to another embodiment, the biometric sensor may be partially arranged on the binding member 212 instead of the housing 211 of the wearable device 101. The arrangement position of the biometric sensor is not limited to the housing 211.

According to an embodiment, the biometric sensor may measure the user's blood pressure by using a photoplethysmography (PPG)-based blood pressure measurement method. The biometric sensor may include a PPG sensor (e.g., a pulse wave sensor). The PPG sensor may emit light to correspond to a predetermined area in which blood vessels of the human body are located, and may receive reflected light of the emitted light to acquire a raw sensor (RAW) signal. For example, the wearable device 101 may identify a variety of biometric information such as heart rate information, stress information, and sleep information of the user based on the RAW signal. According to an embodiment, the wearable device 101 may measure the user's blood pressure based on the RAW signal. For example, the biometric sensor may use a PPG signal (e.g., a RAW signal) to measure blood pressure, and may extract a feature value corresponding to the user's biometric data based on a pulse signal collected through the PPG signal. According to an embodiment, the wearable device 101 may compare the feature value extracted using the biometric sensor with predetermined reference biometric data, and may measure the user's blood pressure based on a difference value according to the comparison result. For example, the biometric sensor may include a light emitting device and a light receiving device. The biometric sensor may emit light through the light emitting device and may receive reflected light of the emitted light through the light receiving device. For example, a part of the light emitted through the light emitting device may be absorbed by the user's blood vessels, and the remaining part thereof may be reflected through the human body. According to an embodiment, at least a part of the light emitted through the light emitting device may be reflected by the human body, and the reflected light may be received through the light receiving device of the biometric sensor. According to an embodiment, the wearable device 101 may acquire biometric data of the user based on the reflected light received through the light receiving device.

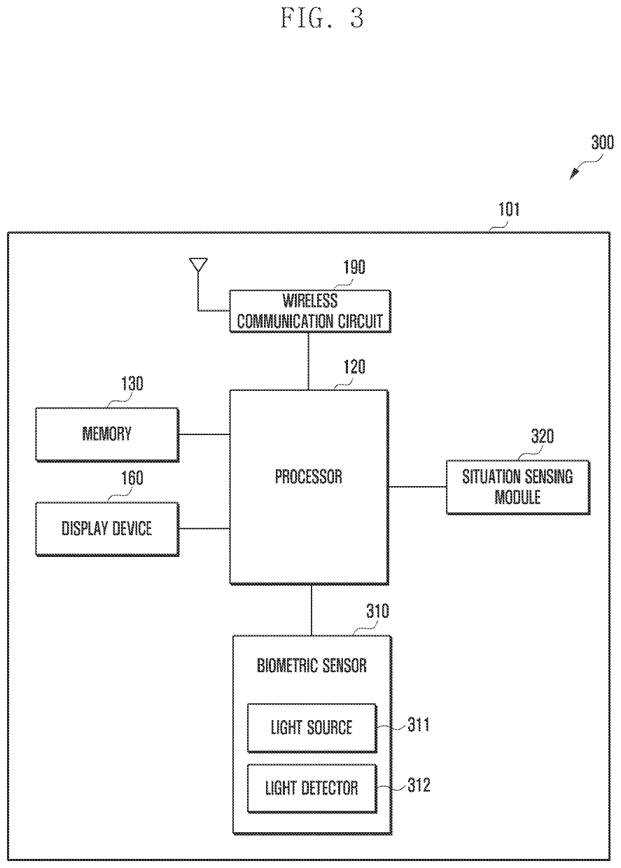

FIG. 3 is a block diagram illustrating an electronic device (e.g., a wearable device) according to an embodiment of the disclosure.

Referring to FIG. 3 depicting block diagram 300, the electronic device 101 (hereinafter, referred to as a wearable device) may include a processor (e.g., the processor 120 of FIG. 1), a memory (e.g., the memory 130 of FIG. 1), a display device (e.g., the display device 160 of FIG. 1), a situation sensing module 320, a wireless communication circuit (e.g., the communication module 190 of FIG. 1), and/or a biometric sensor 310. According to an embodiment, the wearable device 101 may measure blood pressure of the user using the biometric sensor 310. The biometric sensor 310 may include a photoplethysmography (PPG)-based PPG sensor for obtaining user's biometric data.

According to an embodiment, the processor 120 may execute a program (e.g., the program 140 of FIG. 1) stored in the memory 130 to control at least one other component (e.g., a hardware or software component), and may perform various data processing or operations. According to an embodiment, the processor 120 may extract biometric data related to the user using the biometric sensor 310, and may compare the biometric data with reference biometric data (e.g., blood pressure data, first calibrated biometric data, and/or second calibrated biometric data) stored in the memory 130. For example, the blood pressure data may include a user's absolute blood pressure value measured previously (e.g., in calibration operation) using a cuff-type blood pressure monitor. The first calibrated biometric data may refer to biometric data previously (e.g., in calibration operation) measured in a sitting posture, and the second calibrated biometric data may refer to biometric data previously (e.g., in calibration operation) measured in a supine position. According to an embodiment, the reference biometric data may include at least one of the blood pressure data, the first corrected biometric data, and/or the second corrected biometric data. The processor 120 may measure the user's blood pressure in a blood pressure measurement operation by comparing and analyzing the user's biometric data measured in the blood pressure measurement operation and the reference biometric data measured in the calibration operation.

According to an embodiment, the processor 120 may relatively compare and analyze the biometric data measured in the blood pressure measurement operation and the reference biometric data measured in the calibration operation, and may more accurately identify the amount of change in the user's blood pressure at the current measurement time point, compared to the calibration operation.

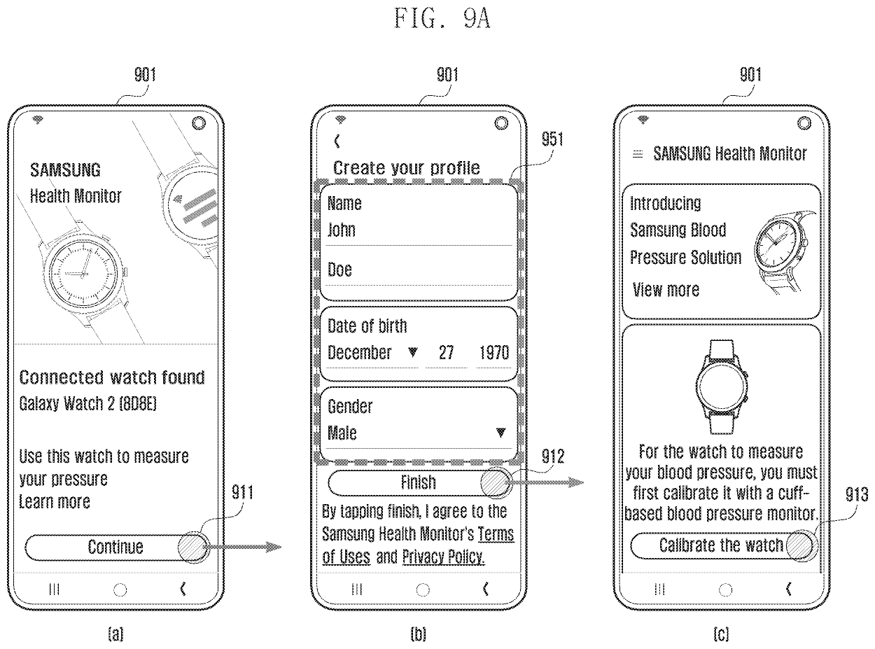

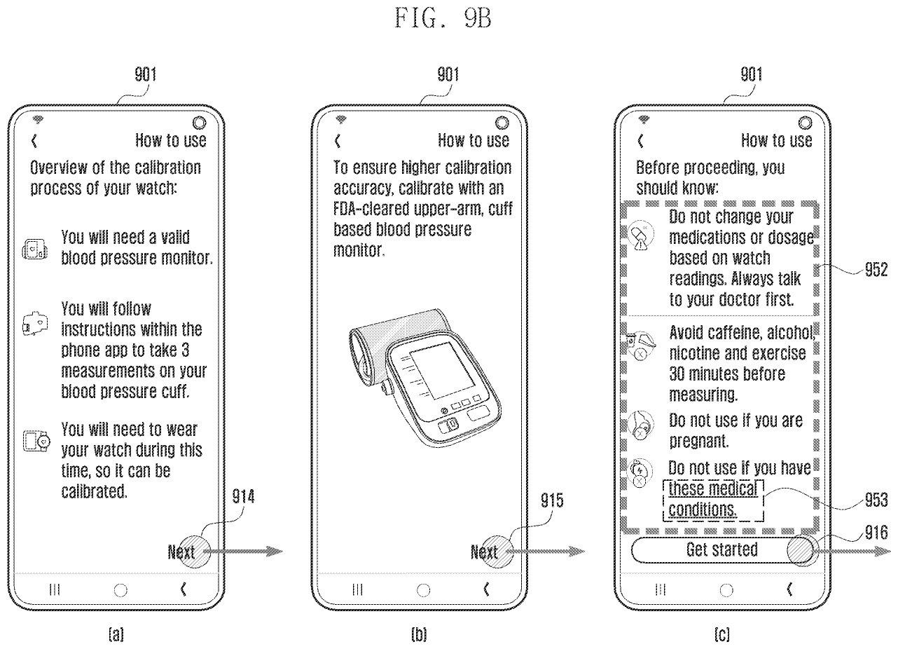

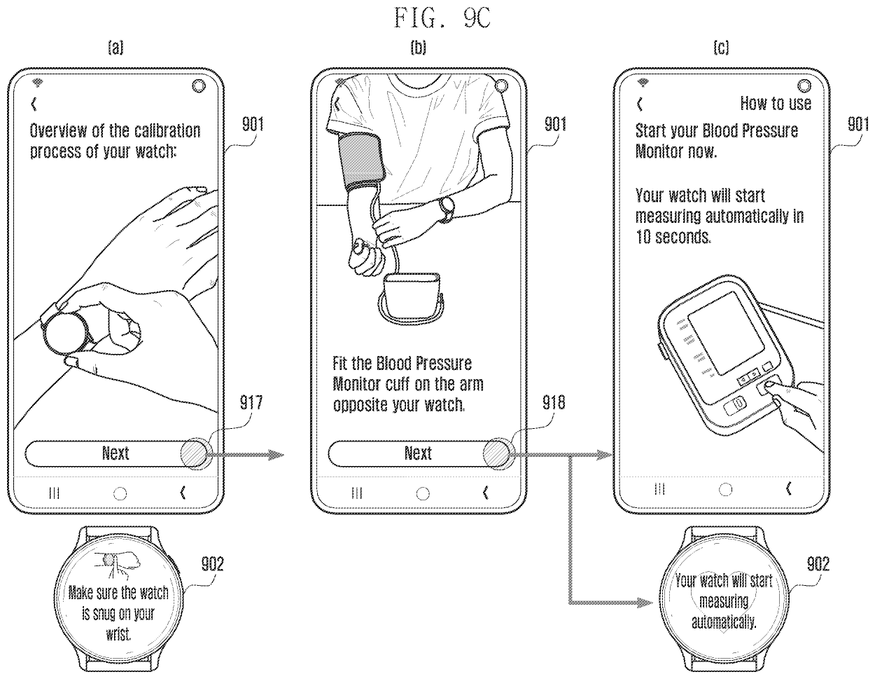

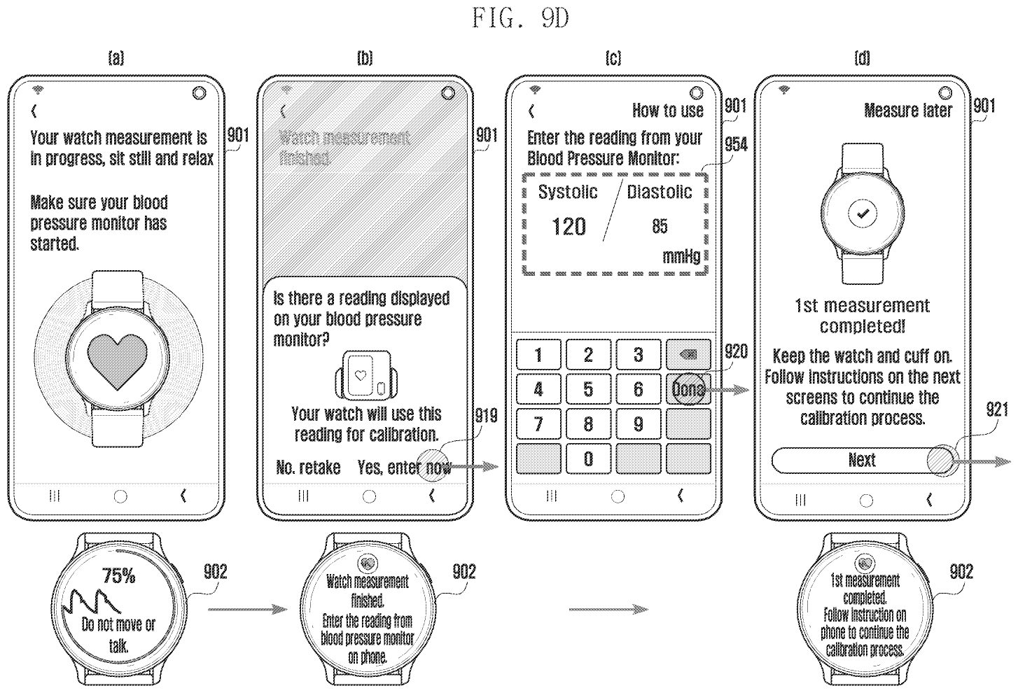

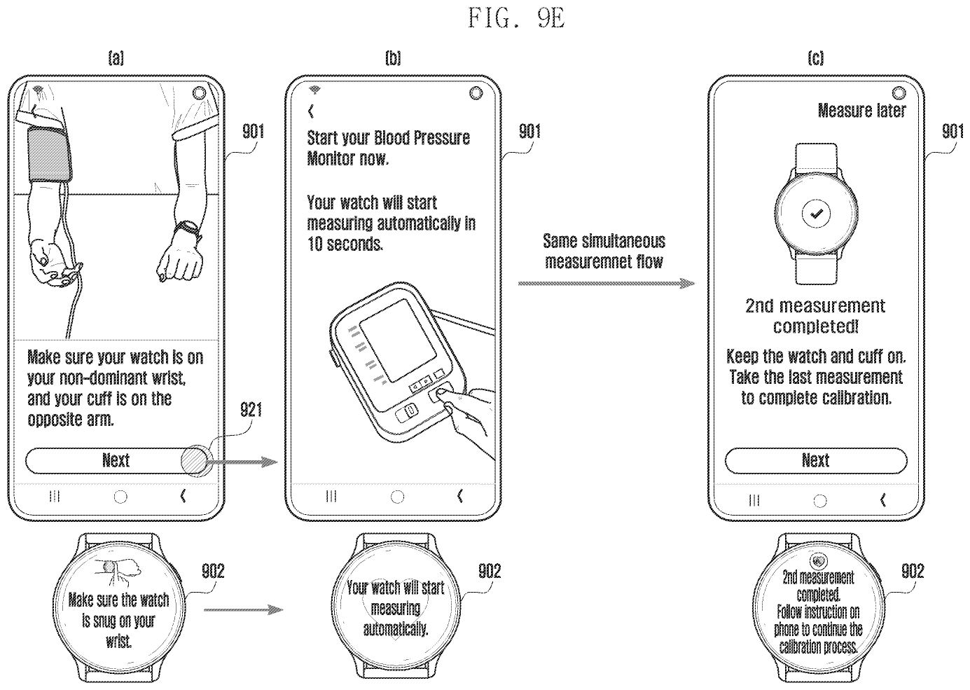

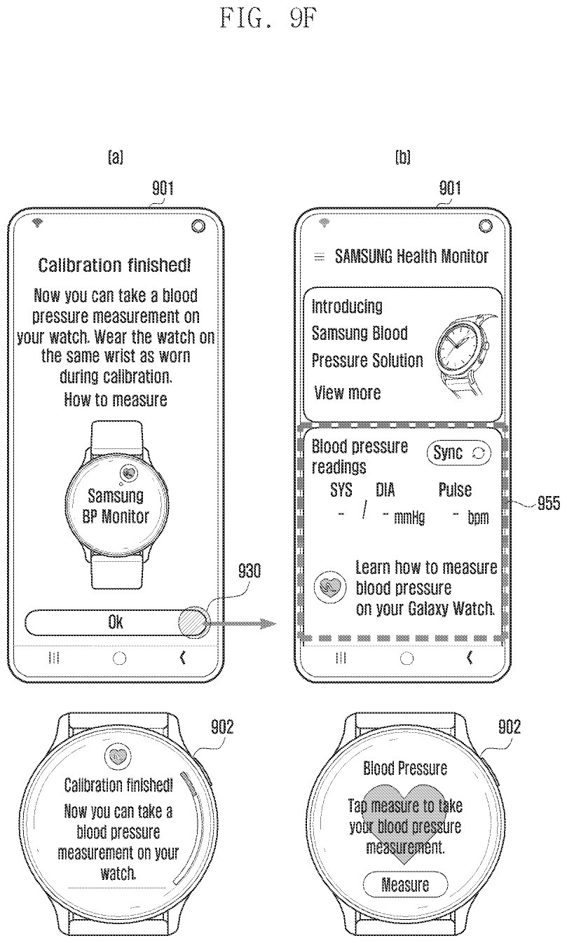

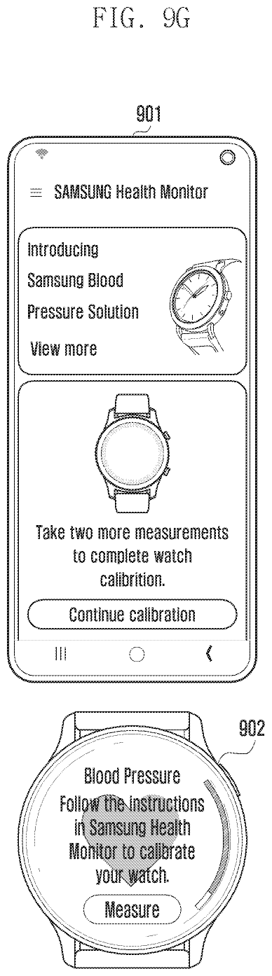

According to an embodiment, the reference biometric data may include at least one of an absolute blood pressure value (e.g., absolute value) measured using a cuff-based blood pressure monitor in the calibration operation and calibrated biometric data measured using a PPG-based sensor (e.g., a PPG sensor and/or the biometric sensor 310) in the calibration operation. The reference biometric data may be determined in the calibration operation and may be stored in the memory 130. According to an embodiment, the biometric data (e.g., calibrated biometric data) may refer to a relative blood pressure (e.g., a relative value) measured using the biometric sensor 310 of the wearable device 101. According to an embodiment, the calibration operation may be an operation of determining reference biometric data serving as a reference value in order to measure the absolute blood pressure of the user in the blood pressure measurement operation. According to an embodiment, the wearable device 101 may periodically update the reference biometric data of the calibration operation in order to more accurately identify the amount of change in the blood pressure. According to an embodiment, in the calibration operation, the absolute blood pressure of the user may be measured using the cuff-type blood pressure monitor in a state in which the user is at rest (e.g., in a stable state), and the calibrated biometric data of the user may be acquired using the biometric sensor 310. According to an embodiment, at substantially the same time point in the calibration operation, the processor 120 may measure the absolute blood pressure and may acquire the calibrated biometric data. Referring to FIGS. 9A to 9G, a process of displaying a user interface (UI) for determining reference biometric data in the calibration operation is illustrated.

According to an embodiment, the reference biometric data may be measured in a state in which the user is at rest. The reference biometric data may include at least one of the user's absolute blood pressure value measured using the cuff-type blood pressure monitor, first calibrated biometric data measured in a sitting position, and/or second calibrated biometric data measured in a supine position. According to an embodiment, the reference biometric data may be stored in the memory 130 using at least one of a method in which a value measured by another electronic device is received through wireless communication, a method in which a user manually input the reference biometric data, and/or a method in which a previously stored value is received through a server.

According to an embodiment, the wearable device 101 may provide a guidance to the user to simultaneously measure the absolute blood pressure and the calibrated biometric data while maintaining in a state where the body and mind of the user are stable in the calibration operation. For example, the wearable device 101 may provide a guidance to the user to maintain a sitting posture in a state where the body and mind of the user are stable, and may acquire first calibrated biometric data based on the guidance. The wearable device 101 may provide a guidance to the user to maintain a supine posture in the state where the body and mind of the user are stable, and may acquire second calibrated biometric data based on the guidance.

According to an embodiment, the wearable device 101 may repeat the blood pressure measurement process for determining the reference biometric data in the calibration operation several times. As the number of repetitions increases, the blood pressure value may be measured more accurately. According to an embodiment, in the calibration operation, the process of measuring blood pressure may be repeated about three times, and an average value of the calibrated biometric data measured about three times may be calculated and determined as the reference biometric data. For example, the wearable device 101 may measure the blood pressure three times in a sitting state in the calibration operation, and may acquire first calibrated biometric data by reflecting a weight value in the sitting state. In the calibration operation, the wearable device 101 may measure the blood pressure three times in a lying state, and may acquire second calibrated biometric data by reflecting a weight value in the lying state. According to another embodiment, when the user speaks or moves in the calibration operation, the calibrated biometric data may be acquired by setting the weight low.

According to an embodiment, in the wearable device 101, before measuring the biometric data at the current time point (e.g., blood pressure measurement operation), the calibration operation may be performed in advance, and the reference biometric data may be in a state of being stored in the memory 130. According to an embodiment, the wearable device 101 may periodically and repeatedly acquire the user's biometric data in the blood pressure measurement operation. The wearable device 101 may extract the user's biometric data in the blood pressure measurement operation, and may more accurately measure the user's blood pressure through the process of comparing and analyzing the user's biometric data with the reference biometric data stored in the memory 130. According to an embodiment, the wearable device 101 may collect at least one PPG signal (e.g., a RAW signal, biometric data) using the biometric sensor 310, and may extract a feature value of a pulse signal based on the collected at least one PPG signal. For example, when the wearable device 101 extracts the biometric data while the user is seated, the wearable device 101 may determine a relative difference value based on the first calibrated biometric data stored in the memory 130 and may calculate the blood pressure of the seated user. When the biometric data is extracted while the user is lying down, the wearable device 101 may determine a relative difference value based on the second calibrated biometric data stored in the memory 130 and may calculate the blood pressure of the user lying down.

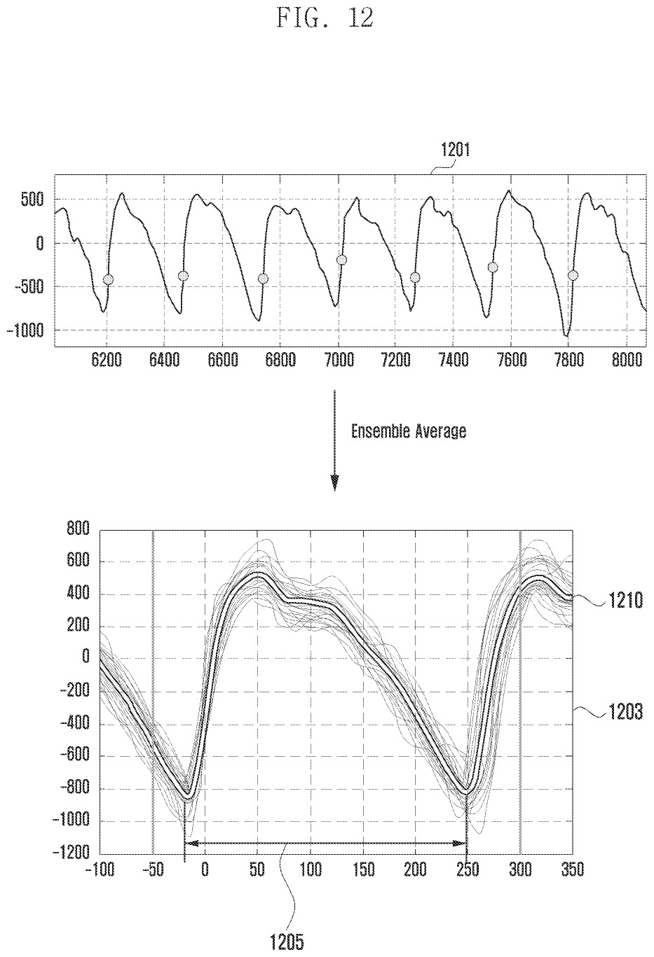

Referring to FIG. 12, a process of measuring user's blood pressure in the wearable device 101 is illustrated. Referring to first graph 1201, the wearable device 101 may acquire at least one PPG signal (e.g., a PPG signal divided based on PPG duration) through the biometric sensor 310, and may calculate an ensemble average based on the acquired at least one PPG signal. Referring to second graph 1203, the wearable device 101 may overlap a plurality of PPG signals to calculate an ensemble average value 1210. According to an embodiment, the wearable device 101 may extract a feature value of the ensemble average value 1210 and may measure the user's blood pressure by comparing the extracted feature value with reference calibrated biometric data stored in the memory 130. According to an embodiment, the wearable device 101 may calculate a PPG duration 1205 from the ensemble average value 1210, and may divide 60 seconds by the PPG duration 1205, thereby measuring a pulse rate corresponding to the user's blood pressure. According to an embodiment, the wearable device 101 may measure the user's blood pressure based on the measured pulse rate.

Pulse rate=60/(one PPG duration in sec) Equation 1

The wearable device 101 may calculate a pulse rate based on Equation 1.

According to an embodiment, the display device 160 of the wearable device 101 may include a screen for displaying the measured biometric data. The display device 160 may include a touch screen capable of sensing a touch. According to an embodiment, the wearable device 101 may display the user's biometric data through the display device 160. According to an embodiment, the wearable device 101 may provide guide information to the user through the display device 160 in the calibration operation, and may display a notification message under a specific condition.

According to an embodiment, the situation sensing module 320 of the wearable device 101 may detect an operating state (e.g., power or temperature) of the wearable device 101 and/or an external environmental state (e.g., user's status information (e.g., sitting state, lying state, speaking state, moving state, and/or apnea state), brightness information and/or time information), and may generate an electrical signal and/or data value corresponding to the sensed state. The situation sensing module 320 may include at least one of an acceleration sensor, a gyro sensor, a proximity sensor, an illumination sensor, a temperature sensor, and/or an audio detection sensor (e.g., an audio module or a microphone). According to an embodiment, the situation sensing module 320 may include at least one sensor for detecting the user's activity state. According to an embodiment, the processor 120 may use the situation sensing module 320 to determine whether the user is sleeping in a snoring state or sleeping in a sleep apnea state (e.g., a sleep disorder state such as severe snoring or daytime sleepiness). According to an embodiment, the wearable device 101 may store information related to user's voice in the memory 130, and may determine whether the user is speaking by using the situation sensing module 320.

According to another embodiment, the situation sensing module 320 of the wearable device 101 may include at least one module for detecting surrounding situation information. For example, the situation sensing module 320 may include at least one of a module for detecting a user's movement, a module for detecting a voice using a microphone, and/or a module for detecting a sleeping state.

According to an embodiment, the wearable device 101 may perform wireless communication with another electronic device (e.g., the electronic devices 102 and 104 of FIG. 1) through a wireless communication circuit (e.g., the communication module 190 of FIG. 1). For example, the wearable device 101 may perform wireless communication with a portable electronic device (e.g., a smartphone), and may exchange commands and/or data (e.g., a speaking state or an apnea state) with each other. According to an embodiment, the wearable device 101 may be at least partially controlled by another external electronic device. For example, the wearable device 101 may perform at least one function under the control of another external electronic device.

According to an embodiment, the biometric sensor 310 of the wearable device 101 may include a photoplethysmography (PPG)-based blood pressure measuring sensor (e.g., a PPG sensor) for extracting user's biometric data. The biometric sensor 310 may include a pulse wave sensor using a PPG signal, and may acquire a raw sensor signal (RAW signal, PPG signal) reflected in response to a predetermined area in which blood vessels of the human body are located. For example, the biometric sensor 310 may include a light emitting device (light source) 311 that emits light and a light receiving device (light detector) (photo detector, PD) 312 that receives the reflected light of the emitted light. The light emitting device 311 and the light receiving device 312 may be arranged to correspond to a sensing area for measuring blood pressure, and may be in a state of being at least partially in contact with the skin where the user's blood vessels are located. The light emitting device 311 may emit light toward the skin where the user's blood vessels are located, and the emitted light may be reflected by the user's skin or blood vessels to generate reflected light. For example, when the flow of blood is large, a lot of light may be absorbed, and when the flow of blood is small, less light may be absorbed. According to an embodiment, the amount of blood flow flowing through the blood vessels may vary over time, and the wearable device 101 may extract the amount of change in the amount of blood flow based on the PPG signal (e.g., reflected light) reflected by the blood vessels. According to an embodiment, the biometric sensor 310 may receive reflected light through the light receiving device 312, and the processor 120 may extract user's biometric data (e.g., cardiac output (CO), total peripheral resistance (TPR), or a biometric parameter) based on the received reflected light (e.g., light scattered or reflected from an area of the skin where the blood vessels are located, PPG signal). For example, the processor 120 may analyze a change in intensity of the reflected light over time. The processor 120 may extract the user's biometric data by analyzing a fluctuation in the reflected light corresponding to a volume change of the user's blood vessels (e.g., blood vessels located on a finger or wrist or a radial camellia below the wrist). For example, the processor 120 may extract the user's biometric data based on a correlation between the amount of change in the reflected light and the volume change. According to an embodiment, the light emitting device 311 and the light receiving device 312 may be configured in plurality. According to an embodiment, the wearable device 101 may detect whether sleep apnea has occurred or measure stress and saturation of percutaneous oxygen (SpO2) of the user, based on the user's biometric data extracted by the PPG signal.

According to various embodiments, the electronic device 101 (e.g., a wearable device) may include the biometric sensor 310 configured to detect biometric data, the situation sensing module 320, the memory 130, and/or the processor 120 configured to be operatively connected to the biometric sensor 310, the situation sensing module 320, and the memory 130. The processor 120 may identify whether a user is in an inactive state using the situation sensing module 320, may identify reference biometric data corresponding to the inactive state when the user is in the inactive state, may extract biometric data of the user using the biometric sensor 310, and may measure the blood pressure of the user based on the extracted biometric data and the reference biometric data stored in the memory 130.

According to an embodiment, the processor 120 may measure a sensing value corresponding to the movement of the electronic device 101 for a predetermined time using an acceleration sensor and a gyro sensor included in the situation sensing module 320, and may identify that the user is in the inactive state when the sensing value corresponding to the movement does not exceed a threshold value.

According to an embodiment, the processor 120 may identify whether the user is in a sleeping state based on at least one of the acceleration sensor, the gyro sensor, and the biometric sensor 310, and may extract the biometric data of the user using the biometric sensor 310 when the user is in the sleeping state.

According to an embodiment, the processor 120 may determine identify the user is sleeping in a sleep apnea state using a microphone included in the situation sensing module 320, and may extract the biometric data of the user to correspond to the sleep apnea state.

According to an embodiment, the processor 120 may identify whether the user is in a speaking state using the microphone included in the situation sensing module 320, and may identify that the user is in the inactive state when the user is not in the speaking state.

According to an embodiment, the processor 120 may acquire context information indicating an external environment using at least one sensor included in the situation sensing module 320, and may map the measured blood pressure and the context information to store the mapped information in the memory 130.

According to an embodiment, the context information may include at least one of location information, posture information, time information, surrounding people information, noise information, health-related information, and sleep phase information.

According to an embodiment, the processor 120 may measure the blood pressure based on the extracted biometric data and the reference biometric data, using a blood pressure measurement algorithm stored in the memory 130.

According to an embodiment, the processor 120 may calculate first reference biometric data based on blood pressure data measured using a cuff blood pressure monitor and a first weight corresponding to a first user posture in a calibration operation, and may calculate second reference biometric data based on the blood pressure data measured using the cuff blood pressure monitor and a second weight corresponding to a second user posture.

According to an embodiment, the processor 120 may provide a guidance corresponding to at least one of the first user posture and the second user posture to the user in the calibration operation, and may calculate at least one of the first reference biometric data and the second reference biometric data based on the guidance.

According to an embodiment, the processor 120 may extract the biometric data of the user acquired using the biometric sensor 310 in a blood pressure measurement operation, may compare the extracted biometric data of the user with at least one of the first reference biometric data and the second reference biometric data to determine a relative difference value corresponding to the extracted biometric data of the user, and may measure the blood pressure of the user based on the determined difference value.

According to an embodiment, the biometric sensor 310 may be a photoplethysmography (PPG)-based blood pressure monitor and may include a light emitting device 311 and a light receiving device 312, and when a part of the light emitted from the light emitting device 311 is absorbed into the user's blood vessels, the remaining part thereof is reflected through the user's body, and the reflected light is received through the light receiving device 312, the processor 120 may extract the biometric data of the user based on the received light.

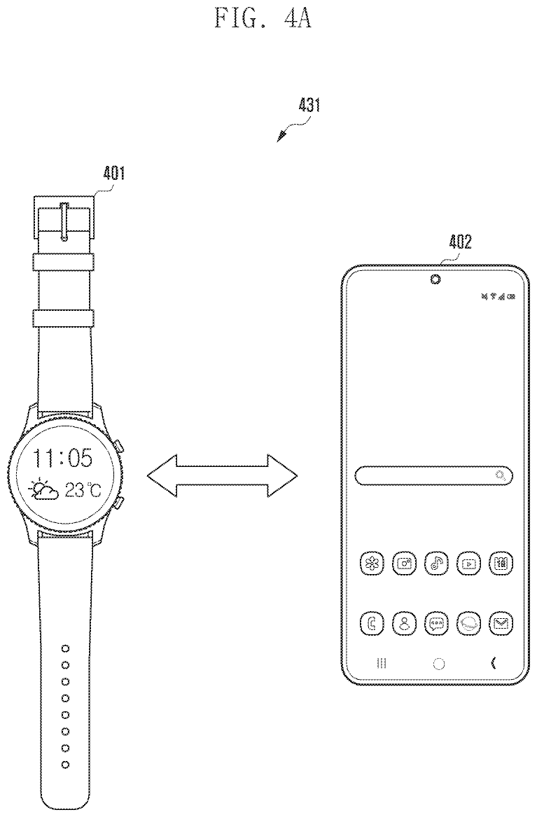

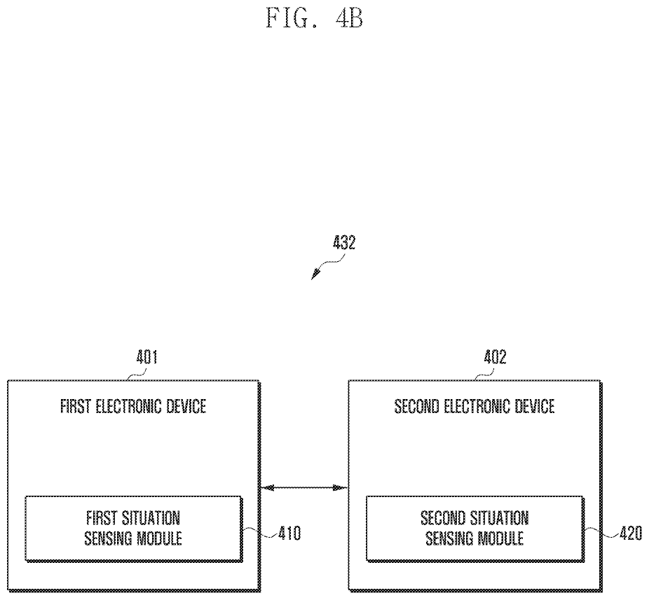

FIG. 4A is a diagram illustrating a case in which a first electronic device (e.g., a wearable device or the electronic device of FIG. 1) is at least partially controlled by a second electronic device 402 (e.g., a portable electronic device) according to an embodiment of the disclosure.

FIG. 4B is a diagram illustrating data exchange between a first electronic device 401 (e.g., a wearable device) and a second electronic device 402 (e.g., a portable electronic device) according to an embodiment of the disclosure.

According to an embodiment, the first electronic device 401 and the second electronic device 402 may be in a state of performing wireless communication, and may be in a state of transmitting and receiving data and commands.

Referring to FIG. 4A, depicting diagram 431, the second electronic device 402 may at least partially control the first electronic device 401. For example, the second electronic device 402 may at least partially control a function performed by the first electronic device 401. According to an embodiment, the first electronic device 401 may include a wearable device provided with a biometric sensor (e.g., a PPG sensor) for extracting user's biometric data. The first electronic device 401 may be provided in such a manner that a sensing area through the biometric sensor directly contacts the human body when the first electronic device 401 is worn by the user. According to an embodiment, the first electronic device 401 and the second electronic device 402 may interwork with each other to execute at least one program at substantially the same time point. For example, the second electronic device 402 may control the first electronic device 401 to extract the user's biometric data by using the biometric sensor. The first electronic device 401 and the second electronic device 402 may be electronic devices including the same components.

Referring to FIG. 4B depicting diagram 432, the first electronic device 401 (e.g., a wearable device or the electronic device 101 of FIG. 1) may perform wireless communication with the second electronic device 402 (e.g., a portable electronic device), and may share data with each other. The first electronic device 401 and the second electronic device 402 may be electronic devices having the same components. According to another embodiment, the first electronic device 401 and the second electronic device 402 may be different types of electronic devices. According to an embodiment, the first electronic device 401 may use a first situation sensing module 410 to detect status information of the user (e.g., a sitting state, a lying state, a speaking state, a moving state, and/or apnea state). The second electronic device 402 may detect the status information of the user by using a second situation sensing module 420. The first electronic device 401 and the second electronic device 402 may exchange and share information related to the surrounding situation and the user's status information.

According to an embodiment, the first situation sensing module 410 may include at least one sensor included in a sensor module (e.g., the sensor module 176 of FIG. 1) of the first electronic device 401 (e.g., the electronic device 101 of FIG. 1). The first electronic device 401 may detect a user's action using the first situation sensing module 410. For example, the first situation sensing module 410 may detect user's activity information (e.g., information related to the user's movement), bedtime information (e.g., information related to whether the user is sleeping), and/or time information (e.g., measurement time information).

According to an embodiment, the second situation sensing module 420 may include at least one sensor included in the sensor module of the second electronic device 402. The second electronic device 402 may detect the user's action and surrounding situation by using the second situation sensing module 420.

According to an embodiment, the first electronic device 401 and the second electronic device 402 may individually detect a user's action, or may interwork with each other to integrally detect the user's action. The first electronic device 401 and the second electronic device 402 may share the detected result data with each other. The first electronic device 401 and the second electronic device 402 may integrate the result data to detect the user's action. According to an embodiment, at least one of the first electronic device 401 and the second electronic device 402 may identify the user's inactive state.

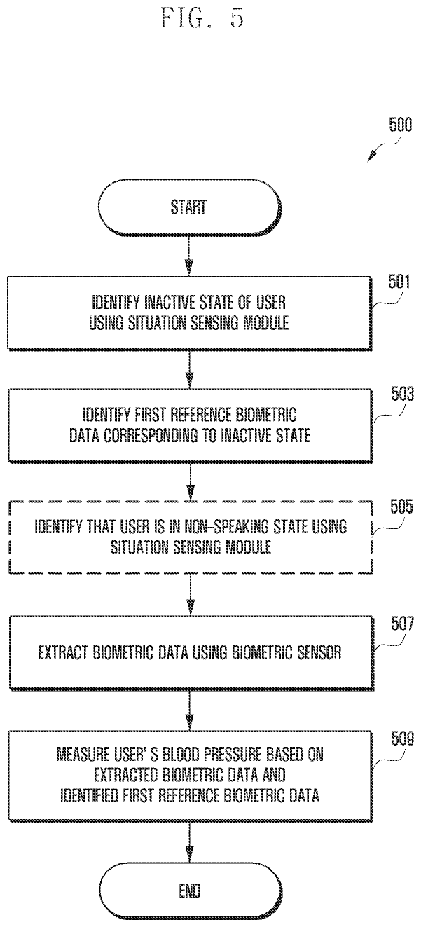

FIG. 5 is a flowchart illustrating a method of measuring blood pressure in the electronic device 101 according to an embodiment of the disclosure.

According to an embodiment, an electronic device (e.g., the electronic device 101 of FIG. 1 or a wearable device) may include a wearable device that maintains a state of being at least partially in contact with the human body when the wearable device is worn by a user. The electronic device 101 may identify an area in which blood vessels of the human body are located as a sensing area, and in response to the sensing area, a biometric sensor (e.g., the biometric sensor 310 of FIG. 3) for acquiring biometric data may be disposed. For example, the electronic device 101 may include a photoplethysmography (PPG)-based biometric sensor (e.g., the biometric sensor 310 of FIG. 3 or a PPG sensor) to measure blood pressure. The electronic device 101 may automatically measure the blood pressure of the user using the biometric sensor every predetermined time, rather than manually measuring the blood pressure according to a user input.

According to an embodiment, the electronic device 101 may identify that the user is in an inactive state using a situation sensing module (e.g., the situation sensing module 320 of FIG. 3) including at least one sensor, and may automatically extract biometric data of the user using the biometric sensor. The electronic device 101 may measure the user's blood pressure by comparing and analyzing the extracted biometric data and reference biometric data stored in a memory (e.g., the memory 130 of FIG. 1).

Referring to FIG. 5 depicting flowchart 500, in operation 501, a processor (e.g., the processor 120 of FIG. 1) of the electronic device 101 may use a situation sensing module (e.g., the situation sensing module 320 of FIG. 3) to identify an inactive state of the user. For example, the inactive state may refer to a state in which the user sits in a chair in a state where the mind and body of the user are stable. According to an embodiment, the processor 120 may use at least one sensor included in the situation sensing module 320 to identify status information of the user (e.g., a sitting state, a lying state, a moving state, and/or or speaking state). The electronic device 101 may identify whether the user is sitting by using the at least one sensor (e.g., an acceleration sensor and/or a gyro sensor) included in the situation sensing module 320. According to an embodiment, the processor 120 may measure a sensing value corresponding to the movement of the electronic device 101 using at least one of the acceleration sensor and/or the gyro sensor. The processor 120 may compare the measured sensing value with a threshold value stored in the memory (e.g., the memory 130 of FIG. 1) for a predetermined time, and may identify that the user is in the inactive state when the sensing value does not exceed the threshold value. According to an embodiment, when the sensing value exceeds the threshold value for a predetermined time, the processor 120 may identify that the user is in an active state, and may stop a blood pressure measurement function.

In operation 503, the processor 120 may identify first reference biometric data corresponding to the inactive state. The first reference biometric data may include first blood pressure data and first calibrated biometric data stored in the memory (e.g., the memory 130 of FIG. 1) in a calibration operation. According to an embodiment, the electronic device 101 may store the first reference biometric data in the calibration operation. For example, the electronic device 101 may measure first blood pressure data of the user using a cuff-type blood pressure monitor in the inactive state, and may store the first blood pressure data as the first reference biometric data. The electronic device 101 may extract first calibrated biometric data of the user using a PPG sensor (e.g., the biometric sensor 310 of FIG. 3) in the inactive state, and may store the extracted first calibrated biometric data as the first reference biometric data. According to an embodiment, the first reference biometric data may include first blood pressure data and first calibrated biometric data of the user who maintains the inactive state. The first blood pressure data may include an absolute blood pressure value of the user, and the first calibrated biometric data may include the user's biometric data based on a PPG signal. According to an embodiment, the electronic device 101 may acquire the first blood pressure data and the first calibrated biometric data in the calibration operation, and may store the first blood pressure data and the first calibrated biometric data in the memory 130 as the first reference biometric data.

In operation 505, the processor 120 may use the situation sensing module 320 to identify whether the user is in a non-speaking state. The situation sensing module 320 may include a microphone and may receive ambient audio. The processor 120 may detect a user's voice based on the received ambient audio. For example, in the electronic device 101, the user's voice information may be stored in the memory 130, and the processor 120 may determine whether the user's voice information is included in the received audio. When the user's voice information is not included in the received audio, the electronic device 101 may identify that the user is not speaking. According to various embodiments, operation 505 may be omitted in the method of measuring blood pressure. Operation 505 may be an additional option for automatically measuring blood pressure.

In operation 507, the processor 120 may extract biometric data using a biometric sensor (e.g., the biometric sensor 310 of FIG. 3). For example, the biometric sensor 310 may include a PPG-based blood pressure measurement sensor (e.g., a PPG sensor and/or a PPG blood pressure monitor). The PPG sensor may extract the user's biometric data using reflected light reflected by the skin where the user's blood vessels are located, and may measure the user's blood pressure based on the extracted biometric data. According to an embodiment, the biometric sensor 310 may include a light emitting device (e.g., the light emitting device 311 of FIG. 3) and a light receiving device (e.g., the light receiving device 312 of FIG. 3), and may emit light through the light emitting device 311 and receive light through the light receiving device 312. In the biometric sensor 310, a sensing area for measuring the user's blood pressure may be provided in response to the area in which the user's blood vessels are located. According to an embodiment, the processor 120 may emit light using the light emitting device 311, and the emitted light may be reflected by the skin where the user's blood vessels are located to generate reflected light. The processor 120 may acquire the reflected light using the light receiving device 312. For example, the reflected light may include a PPG signal from which the user's biometric data can be extracted. According to an embodiment, the processor 120 may extract the user's biometric data based on the acquired reflected light (e.g., a PPG signal).

In operation 509, the processor 120 may measure the user's blood pressure based on the extracted biometric data and the first reference biometric data stored in the memory (e.g., the memory 130 of FIG. 1). For example, the first reference biometric data may include the user's blood pressure data (e.g., absolute value) and the first calibrated biometric data. According to an embodiment, the processor 120 may compare the extracted biometric data with the first calibrated biometric data to determine a relative difference value, and may measure the user's blood pressure based on the relative difference value. The processor 120 may measure the user's blood pressure value in the blood pressure measurement operation by reflecting the relative difference value in the user's blood pressure data (e.g., absolute value). According to an embodiment, the first calibrated biometric data may include the biometric data extracted from the user who is in the inactive state (e.g., a sitting state) in the calibration operation. According to an embodiment, in a state in which a blood pressure measurement algorithm for measuring the user's blood pressure is stored in the memory 130, the processor 120 may reflect the extracted biometric data and the first reference biometric data to the blood pressure measurement algorithm. The processor 120 may measure the user's blood pressure based on the blood pressure measurement algorithm.

According to an embodiment, the electronic device 101 may provide a guidance for measuring the blood pressure in a sitting posture to the user whose mind and body is stable in the calibration operation. The electronic device 101 may acquire the first blood pressure data (e.g., absolute value) and biometric data (e.g., first calibrated biometric data) measured based on the guidance. According to an embodiment, the first reference biometric data may include the user's first blood pressure data measured using the cuff blood pressure monitor in the calibration operation, and the user's first calibrated biometric data measured using the biometric sensor (e.g., PPG sensor or PPG blood pressure monitor). For example, the cuff blood pressure monitor may measure the user's blood pressure data (e.g., a systolic blood pressure value and/or a diastolic blood pressure value) corresponding to an absolute value. According to an embodiment, the electronic device 101 may display a user interface for receiving the blood pressure data measured using the cuff blood pressure monitor, and may store the blood pressure data input by the user as the first reference biometric data in the memory 130.

According to an embodiment, the electronic device 101 may extract biometric data of the user corresponding to the current time in the blood pressure measurement operation. According to an embodiment, the electronic device 101 may extract the user's biometric data using the biometric sensor 310 (e.g., a PPG sensor) in the blood pressure measurement operation, and may compare the extracted biometric data with the first reference biometric data stored in the memory 130, thereby measuring the user's blood pressure.

According to an embodiment, the electronic device 101 may use the situation sensing module 320 to identify the inactivity state and non-speaking state of the user, and may use the biometric sensor 310 to extract the biometric data in the current time point. The electronic device 101 may compare and analyze the biometric data at the current time point with the first reference biometric data stored in the memory 130 to measure the user's blood pressure. According to an embodiment, when a specific condition (e.g., a condition in which the user maintains an inactive state for a predetermined time) is satisfied in daily life, the electronic device 101 may automatically extract the user's biometric data and may measure and record the blood pressure value of the user based on the extracted biometric data. According to an embodiment, the electronic device 101 may detect user's status information (context information), and may measure and record the blood pressure value based on the status information.

According to an embodiment, the electronic device 101 may compare the first calibrated biometric data measured using the biometric sensor 310 in the calibration operation with the biometric data measured using the biometric sensor 310 in the blood pressure measurement operation, and may identify whether a difference value of the comparison exceeds a predetermined threshold value. For example, when the difference value exceeds the threshold value, the electronic device 101 may identify that the measured first biometric data is erroneous data and may not store the first biometric data in the memory 130.

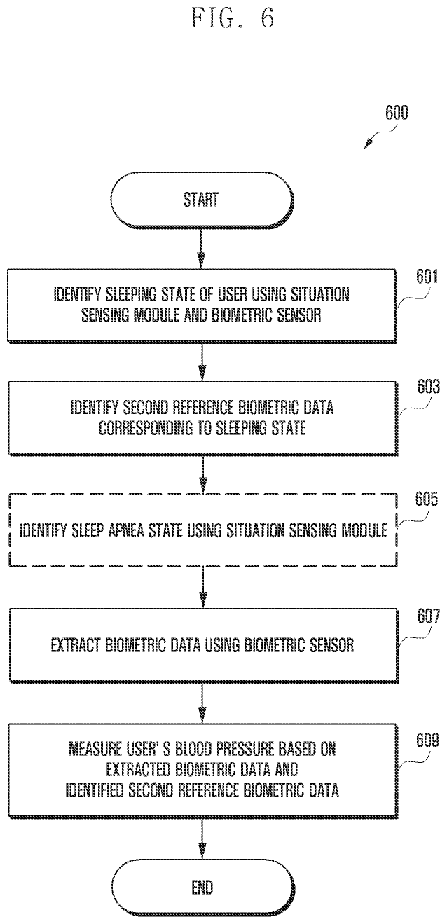

FIG. 6 is a flowchart illustrating a method of measuring blood pressure by detecting a sleeping state by the electronic device 101 according to an embodiment of the disclosure.

Referring to FIG. 6 depicting flowchart 600, in operation 601, a processor (e.g., the processor 120 of FIG. 1) of an electronic device (e.g., the electronic device 101 of FIG. 1) may identify a sleeping state of a user using at least one of a situation sensing module (e.g., the situation sensing module 320 of FIG. 3) and a biometric sensor (e.g., the biometric sensor 310 of FIG. 3). For example, identifying the sleeping state may refer to identifying whether the user is sleeping. The electronic device 101 may identify whether the user is sleeping by using at least one sensor (e.g., an acceleration sensor and/or a gyro sensor) included in the situation sensing module 320 and the biometric sensor 310. In general, the blood pressure of a person is lowered at bedtime than in a state of daily living. For example, the blood pressure generally begins to rise just before waking up, continues to rise for about 1 to 2 hours, maintains during daily life, and falls at bedtime. According to an embodiment, the processor 120 may extract the user's biometric data using the biometric sensor 310 and may identify the user's sleeping state based on the extracted biometric data.

In operation 603, the processor 120 may identify second reference biometric data corresponding to the sleeping state. The second reference biometric data may include second blood pressure data and second calibrated biometric data stored in a memory (e.g., the memory 130 of FIG. 1) in the calibration operation. According to an embodiment, the electronic device 101 may store the second reference biometric data in the calibration operation. For example, the electronic device 101 may measure the user's second blood pressure data using a cuff-type blood pressure monitor in a lying state, and may reflect a weight to the second blood pressure data to obtain blood pressure data corresponding to the sleeping state. The electronic device 101 may store the blood pressure data corresponding to the sleeping state as the second reference biometric data. The electronic device 101 may extract second calibrated biometric data of the user using a PPG sensor (e.g., the biometric sensor 310 of FIG. 3) in the lying state, and may reflect a weight to the second calibrated biometric data to calculate calibrated biometric data corresponding to the sleeping state. The electronic device 101 may store the calibrated biometric data corresponding to the sleeping state as the second reference biometric data. According to an embodiment, the second reference biometric data may include second blood pressure data and second calibrated biometric data of the user who maintains the sleeping state. The second blood pressure data may include an absolute blood pressure value of the user, and the second calibrated biometric data may include the user's biometric data based on a PPG signal. According to an embodiment, the electronic device 101 may acquire the second blood pressure data and the second calibrated biometric data in the calibration operation, and may store the second blood pressure data and the second calibrated biometric data in the memory 130 as the second reference biometric data.

In operation 605, the processor 120 may identify whether the user is in a sleep apnea state using the situation sensing module 320. The situation sensing module 320 may include a microphone and may receive ambient audio. The processor 120 may detect whether the sleeping user is in the sleep apnea state (e.g., a sleep disorder state such as severe snoring or daytime sleepiness) based on the received ambient audio. According to various embodiments, operation 605 may be omitted in the method of measuring the blood pressure. Operation 605 may be an additional option for automatically measuring the blood pressure.

In operation 607, the processor 120 may extract biometric data using the biometric sensor 310. For example, the biometric sensor 310 may include a PPG-based blood pressure measurement sensor (e.g., a PPG sensor and/or a PPG blood pressure monitor). The PPG sensor may extract the user's biometric data using reflected light reflected by the skin where the user's blood vessels are located, and may measure the user's blood pressure based on the extracted biometric data. The process of extracting the user's biometric data is the same as operation 507 of FIG. 5, and since the process of extracting the user's biometric data has been described above, the above description is substituted therefor.

In operation 609, the processor 120 may measure the user's blood pressure based on the extracted biometric data and second reference biometric data stored in the memory (e.g., the memory 130 of FIG. 1). For example, the second reference biometric data may include user's blood pressure data (e.g., absolute value) and second calibrated biometric data. According to an embodiment, the processor 120 may compare the extracted biometric data with the second calibrated biometric data to identify a relative difference value, and may measure the user's blood pressure based on the relative difference value. The processor 120 may measure the user's blood pressure value in the blood pressure measurement operation by reflecting the relative difference value in the user's blood pressure data (e.g., absolute value). According to an embodiment, the second calibrated biometric data may include calibrated biometric data in which a weight is reflected in the biometric data extracted from the user lying down in the calibration operation.

According to an embodiment, the electronic device 101 may provide a guidance for measuring blood pressure in a supine position to the user whose mind and body is stable in the calibration operation. The electronic device 101 may acquire second blood pressure data (e.g., absolute value) and biometric data (e.g., second calibrated biometric data) measured based on the guidance. According to an embodiment, the second reference biometric data may include the user's second blood pressure data measured using the cuff blood pressure monitor in the calibration operation and the user's second blood pressure data measured using the biometric sensor (e.g., a PPG sensor and/or a PPG blood pressure monitor). For example, the cuff blood pressure monitor may measure the user's blood pressure data (e.g., a systolic blood pressure value and/or a diastolic blood pressure value) corresponding to an absolute value. According to an embodiment, when acquiring the second reference biometric data, the electronic device 101 may calculate the calibrated biometric data corresponding to the sleeping state by reflecting a weight to the second calibrated biometric data measured in the supine position. According to an embodiment, the processor 120 may measure the user's blood pressure in the sleeping state by comparing the extracted biometric data with the second reference biometric data.

According to an embodiment, the electronic device 101 may store the user's second calibrated biometric data measured in a lying state in the memory 130. For example, there may be a difference between the biometric data measured in the lying state and the biometric data measured in the sleeping state. According to an embodiment, the wearable device 101 may calculate the calibrated biometric data corresponding to the sleeping state by reflecting a weight to the second calibrated biometric data measured in the lying state. The electronic device 101 may calculate the second reference biometric data in the calibration operation and store the second reference biometric data in the memory 130.

According to various embodiments, the electronic device 101 may use the situation sensing module 320 to identify the user's sleeping state, and may use the biometric sensor 310 to extract the biometric data in the sleeping state. The electronic device 101 may compare and analyze the extracted biometric data and the second calibrated biometric data stored in the memory 130 to identify a relative difference value, and may measure the user's blood pressure based on the relative difference value. According to an embodiment, when a specific condition (e.g., a condition in which the user maintains a sleeping state for a predetermined time) is satisfied during sleep, the electronic device 101 may automatically extract the user's biometric data, and may measure and record the user's blood pressure value based on the extracted biometric data. According to an embodiment, the electronic device 101 may detect user's status information (context information), and may measure and record the blood pressure value based on the status information.

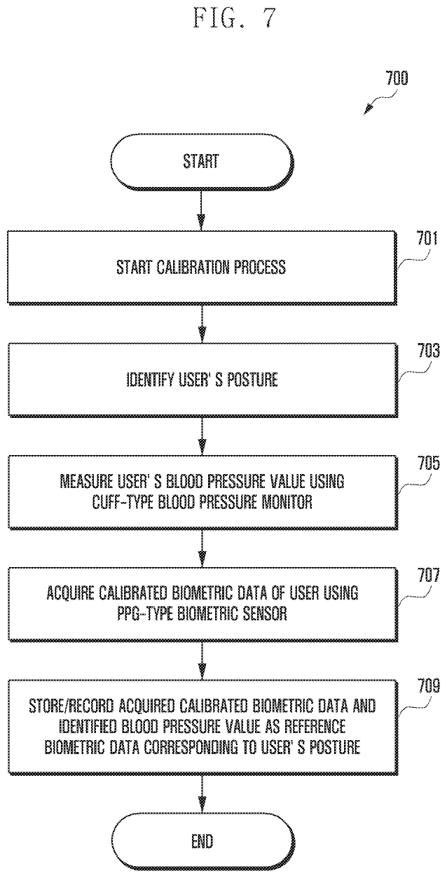

FIG. 7 is a flowchart illustrating a method of recording reference biometric data in a calibration operation according to an embodiment of the disclosure.

According to an embodiment, the electronic device (e.g., the electronic device 101 of FIG. 1) may store reference biometric data in a memory (e.g., the memory 130 of FIG. 1) in a calibration operation, and may extract user's biometric data in a blood pressure measurement operation, and may compare and analyze the extracted biometric data with the reference biometric data stored in the memory 130. According to an embodiment, the electronic device 101 may measure a user's blood pressure value in the blood pressure measurement operation through the comparison and analysis.

Referring to FIG. 7 depicting flowchart 700, in operation 701, the electronic device 101 may start a calibration process. For example, the calibration process may be an operation of storing reference biometric data in the memory 130 to more accurately measure the user's blood pressure value.

In operation 703, the processor of the electronic device 101 (e.g., the processor 120 of FIG. 1) may identify the posture of the user. For example, the processor 120 may identify the posture of the user through a situation sensing module (e.g., the situation sensing module 320 of FIG. 3) or may provide a guidance to the user to maintain a specific posture of the user.

In operation 705, the processor 120 may measure the user's blood pressure value using a cuff-type blood pressure monitor. For example, the cuff-type blood pressure monitor may include an external electronic device. The user may measure the blood pressure value corresponding to an absolute value through the cuff-type blood pressure monitor. According to another embodiment, the electronic device 101 may be functionally connected to the cuff-type blood pressure monitor, and may control the cuff-type blood pressure monitor to measure the user's blood pressure value. According to an embodiment, the electronic device 101 may separate and store a first blood pressure value measured in a sitting posture of the user and a second blood pressure value measured in a supine posture of the user.

In operation 707, the processor 120 may acquire the user's calibrated biometric data using a PPG-type biometric sensor. According to an embodiment, the electronic device 101 may separate and store the first calibrated biometric data measured in the user's sitting posture and the second calibrated biometric data measured in the user's supine posture. According to an embodiment, the electronic device 101 may perform operations 705 and 707 substantially simultaneously to measure a more accurate blood pressure value.

In operation 709, the processor 120 may store the acquired calibration biometric data and the identified blood pressure value as the reference biometric data corresponding to the user's posture. According to an embodiment, the electronic device 101 may store the first blood pressure value and the first calibrated biometric data as first reference biometric data, and may store the second blood pressure value and the second calibrated biometric data as second reference biometric data. For example, the first reference biometric data may include reference biometric data measured by performing a calibration process in the user's sitting posture. For example, the second reference biometric data may include the reference biometric data measured by performing the calibration process in the user's supine position. According to an embodiment, the electronic device 101 may calculate the reference biometric data corresponding to the sleeping state by reflecting a weight in the second reference biometric data.

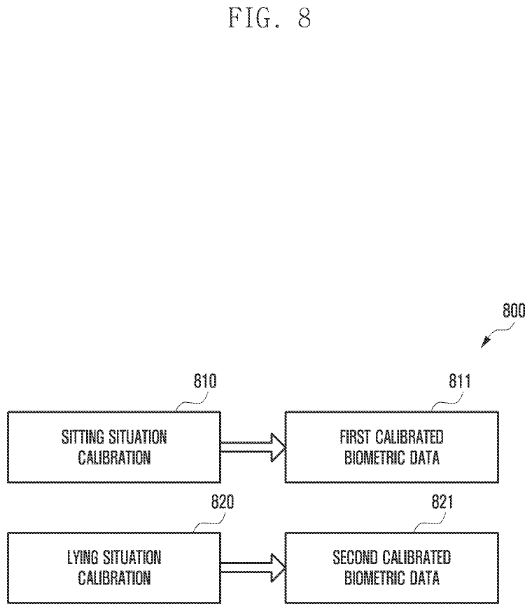

FIG. 8 is a diagram illustrating a process of acquiring reference biometric data (e.g., first reference biometric data and/or second reference biometric data) in response to a user's posture according to an embodiment of the disclosure.

According to an embodiment, when measuring user's blood pressure using a biometric sensor (e.g., the biometric sensor 310 and the PPG sensor of FIG. 3), an electronic device (e.g., the electronic device 101 of FIG. 1) may measure and record reference biometric data in a calibration operation to more accurately measure the blood pressure. According to an embodiment, the reference biometric data may include a blood pressure value (e.g., an absolute value) measured using a cuff blood pressure monitor and calibrated biometric data extracted using a PPG sensor. The electronic device 101 may acquire the reference biometric data based on the blood pressure value and the calibrated biometric data in the calibration operation.

Referring to FIG. 8 depicting diagram 800, according to an embodiment, the electronic device 101 may perform calibration operation 810 on the user while the user is in a sitting state with a stable mind and body, and may collect first calibrated biometric data 811. For example, the electronic device 101 may provide a guidance to the user to measure the blood pressure in a sitting position, and may collect the first calibrated biometric data 811 from the user based on the guidance. According to an embodiment, the electronic device 101 may extract first reference biometric data based on the first blood pressure value measured using the cuff blood pressure monitor in the user's sitting state and the first calibrated biometric data 811 extracted using the biometric sensor 310 (PPG sensor) in the user's sitting state.

According to an embodiment, the electronic device 101 may perform calibration operation 820 on the user who is in a lying state with a stable mind and body, and may collect second calibrated biometric data 821. For example, the electronic device 101 may provide a guidance to the user to measure the blood pressure in a supine position, and may collect the second calibrated biometric data 821 from the user based on the guidance. According to an embodiment, the electronic device 101 may acquire second reference biometric data based on a second blood pressure value measured using the cuff blood pressure monitor in the user's lying state and the second calibrated biometric data 821 extracted using the biometric sensor 310 (PPG sensor) in the user's lying state.

According to an embodiment, the electronic device 101 may repeat a process for acquiring the reference biometric data a plurality of times in the calibration operation, and as the number of repetitions increases, the accuracy of blood pressure measurement may increase. For example, the electronic device 101 may repeat the measurement of the blood pressure value using the cuff blood pressure monitor and the extraction of the calibrated biometric data using the PPG sensor about three times, and may calculate an average value of the blood pressure value and calibrated biometric data measured about three times to determine the reference biometric data. According to an embodiment, the electronic device 101 may perform calibration operation 810 in the sitting posture and may acquire the first calibrated biometric data 811 by reflecting a weight corresponding to the sitting posture. According to an embodiment, the electronic device 101 may perform calibration operation 820 in the supine posture, and may acquire the second calibrated biometric data 821 by reflecting a weight corresponding to the supine posture. The electronic device 101 may acquire the reference biometric data including at least one of the blood pressure value measured using the cuff blood pressure monitor in the calibration operation, the first calibrated biometric data 811, and/or the second calibrated biometric data 821.

FIGS. 9A, 9B, 9C, 9D, 9E, 9F, and 9G are first to seventh diagrams illustrating a user interface displayed in a calibration operation according to various embodiments of the disclosure.

Referring to FIGS. 9A to 9G, a portable electronic device 901 (e.g., the second electronic device 402 of FIG. 4A) may operate in conjunction with a wearable device 902 (e.g., the first electronic device 401 of FIG. 4A), and may at least partially control the operation of the wearable device 902. Referring to FIGS. 9A to 9G, a procedure for performing a calibration process in the portable electronic device 901 is illustrated, but is not limited to the portable electronic device 901. According to another embodiment, the wearable device 902 may independently perform the calibration process.

Referring to FIG. 9A, in part [a], an application program (e.g., SAMSUNG Health Monitor) for measuring user's blood pressure is executed in the portable electronic device 901, and an execution screen of the application program is displayed on a display. In part [a] of FIG. 9A, the portable electronic device 901 may search for a functionally connectable wearable device 902 and may display a model name of the wearable device 902. In response to a user input 911 in part [a] of FIG. 9A, the portable electronic device 901 may display the screen of part [b] of FIG. 9A for obtaining user's profile information 951. For example, the user's profile information 951 may include personal information such as the user's name, date of birth, and/or gender, and health-related information. According to an embodiment, when the application program is executed, the portable electronic device 901 may identify a health identification (ID) of the user (e.g., resident registration number, medical insurance number, national registration number, and/or user's unique ID information), and may store the profile information 951 of the user based on the health ID. According to another embodiment, the portable electronic device 901 may be functionally connected to a server of a medical institution managed by the nation, and may store the user's health ID and profile information 951 in the database of the medical institution server. According to an embodiment, the portable electronic device 901 may utilize the input user profile information 951 in various programs. In response to a user input 912 in part [b] of FIG. 9A, the portable electronic device 901 may store the user's profile information 951 and may enter part [c] of FIG. 9A for performing a calibration process. In response to the user input 913 in part [c] of FIG. 9A, the portable electronic device 901 may be synchronized with the functionally connected wearable device 902, and the portable electronic device 901 and the wearable device 902 may perform the calibration process substantially at the same time.

Referring to FIG. 9B, in part [a], the portable electronic device 901 may display content to be noted before performing the calibration process. In response to the user input 914 in part [a] of FIG. 9B, the portable electronic device 901 may display how to use an external cuff blood pressure monitor. According to another embodiment, the portable electronic device 901 may be functionally connected to the cuff blood pressure monitor, and may at least partially control the cuff blood pressure monitor. In response to the user input 915 in part [b] of FIG. 9B, the portable electronic device 901 may display a notice 952 corresponding to the user based on the user's profile information 951. For example, medical information based on the user's health information may be displayed, or content to be noted according to the user's age and gender may be displayed. For example, the portable electronic device 901 may display an object 953 for displaying detailed information related to the user's health, and in response to a user input for the object 953, specific health information corresponding to the user may be displayed. According to an embodiment, the portable electronic device 901 may identify a communication service provider for wireless communication, and may identify country information in which the portable electronic device 901 is located. The portable electronic device 901 may display a policy for blood pressure measurement based on the country information. In response to the user input 916 in part [c] of FIG. 9B, the portable electronic device 901 may display the screen of FIG. 9C.

Referring to FIG. 9C, in part [a], the portable electronic device 901 may be functionally connected to the wearable device 902 and may at least partially control the wearable device 902. According to an embodiment, the portable electronic device 901 and the wearable device 902 may perform a calibration process substantially at the same time. In response to a user input 917 in part [a] of FIG. 9C, the portable electronic device 901 may guide the measurement of the blood pressure value using the cuff blood pressure monitor. In response to a user input 918 in part [b] of FIG. 9C, the portable electronic device 901 may perform measurement of the blood pressure value using the cuff blood pressure monitor and extraction of the biometric data using the wearable device 902 substantially at the same time. According to an embodiment, in part [c] of FIG. 9C, the portable electronic device 901 may control the wearable device 902 to automatically extract the user's biometric data from the wearable device 902.

Referring to FIG. 9D, in part [a], the wearable device 902 may display the progress of a biometric data extraction process. In part [b] of FIG. 9D, the portable electronic device 901 may display a user interface for identifying whether the measurement of the blood pressure value using the cuff blood pressure monitor is completed. In part [b] of FIG. 9D, the wearable device 902 may indicate that the extraction of the biometric data is completed. In response to a user input 919 in part [b] of FIG. 9D, the portable electronic device 901 may display a user interface 954 for receiving the measured blood pressure value from the user. For example, the user may separately input a systolic blood pressure value and a diastolic blood pressure value. In response to a user input 920 in part [c] of FIG. 9D, the portable electronic device 901 may indicate that a first calibration process has been completed. According to an embodiment, the calibration process may be repeatedly performed several times, and as the number of repetitions increases, the accuracy of the measured blood pressure value may increase. In response to a user input 921 in part [d] of FIG. 9D, the portable electronic device 901 may display the screen of FIG. 9E.

Referring to FIG. 9E, parts [a] and [b] may be the same as parts [a] to [c] of FIG. 9C. FIG. 9E illustrates screens displayed by the portable electronic device 901 and the wearable device 902 in a second calibration process. In part [d] of FIG. 9D, the portable electronic device 901 and the wearable device 902 may indicate that the second calibration process has been completed.

According to an embodiment, the portable electronic device 901 may perform a calibration process while maintaining the same posture (e.g., a sitting posture, a standing posture, and/or a lying posture). The portable electronic device 901 may repeat the calibration process for a predetermined number of times (e.g., three times). According to an embodiment, the portable electronic device 901 may use a situation sensing module (e.g., the situation sensing module 320 of FIG. 3) to identify the posture of the user. For example, when the calibration process is performed, the portable electronic device 901 may detect that the user's posture has changed, and in response to the detection, the portable electronic device 901 may stop the currently performed calibration process. The portable electronic device 901 may notify the user that the posture has been changed. According to an embodiment, when the user's posture is changed, the portable electronic device 901 may delete the measurement data of the calibration process being performed. According to an embodiment, when performing the calibration process, the portable electronic device 901 may identify the user's posture using the situation sensing module 320 and may apply a weight corresponding to the identified user's posture to the calibration process. The portable electronic device 901 may acquire reference biometric data to which the weight corresponding to the user's posture is reflected.

According to another embodiment, when performing the calibration processes multiple times, the portable electronic device 901 may perform the calibration process while changing the posture. For example, when performing the calibration process for a predetermined number of times (e.g., 3 times), the portable electronic device 901 may perform the calibration process based on the sitting posture during the first measurement, and may perform the calibration process based on different postures (e.g., a standing posture or a lying posture) during the second and third measurement. According to another embodiment, the portable electronic device 901 may perform the calibration process based on the same posture for a predetermined number of times (e.g., 3 times), and may additionally (e.g., 4 times or more) perform the calibration process based on different postures.

Referring to FIG. 9F, in part [a], the portable electronic device 901 and the wearable device 902 may indicate that the calibration process has been completed. In response to a user input 930 in part [b] of FIG. 9F, the portable electronic device 901 and the wearable device 902 may periodically measure the user's blood pressure, and may list 955 and record the measured user's blood pressure. According to an embodiment, the portable electronic device 901 and the wearable device 902 may automatically measure the user's blood pressure value based on context information. For example, the wearable device 902 may extract the user's biometric data using the PPG sensor, and may measure the user's blood pressure value through comparison with the user's calibrated biometric data determined in the calibration process. According to an embodiment, the portable electronic device 901 and the wearable device 902 may store the measured blood pressure value together with the context information in the memory.

Referring to FIG. 9G, the portable electronic device 901 may perform the calibration process again. For example, the portable electronic device 901 may perform the calibration process only once or twice while performing the calibration process for a predetermined number of times (e.g., 3 times), and may not complete the calibration process. Referring to FIG. 9G, the portable electronic device 901 may display a user interface for completing an incomplete calibration process. According to an embodiment, the portable electronic device 901 may be functionally connected to the wearable device 902 to at least partially control the wearable device to perform the same process. According to an embodiment, the portable electronic device 901 may use the situation sensing module (e.g., the situation sensing module 320 of FIG. 3) to identify the user's posture, and may stop the currently performed calibration process when a change in user's posture is detected. Referring to FIG. 9G, the portable electronic device 901 may display the user interface of FIG. 9G even when the calibration process is stopped. According to an embodiment, the portable electronic device 901 and the wearable device 902 may configure a notification event to periodically perform the calibration process.

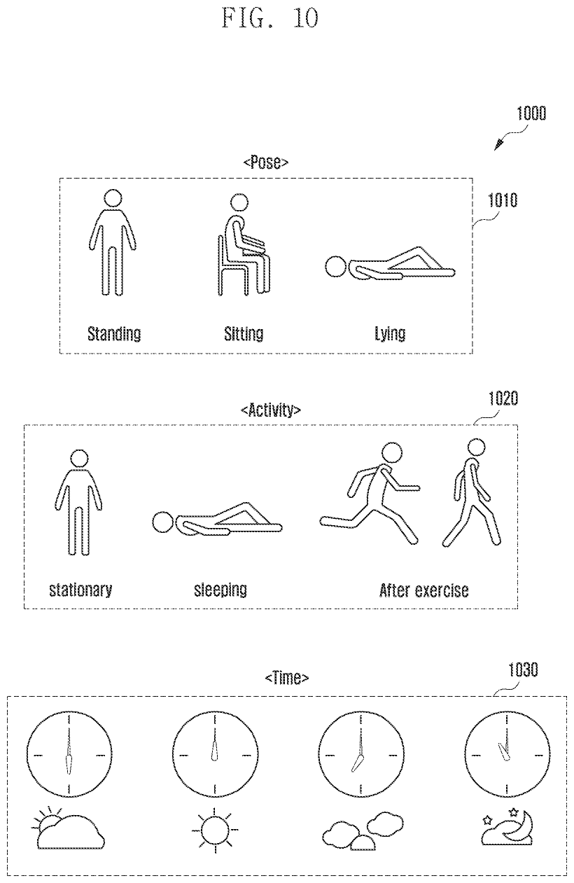

FIG. 10 is a diagram illustrating a plurality of pieces of context information that can be detected by the electronic device 101 according to an embodiment of the disclosure.

According to an embodiment, an electronic device (e.g., the electronic device 101 of FIG. 1) may use a situation sensing module (e.g., the situation sensing module 320 of FIG. 3) to collect context information (e.g., status information) indicating an external environment.

Referring to FIG. 10 depicting diagram 1000, for example, the context information may include information 1010 related to a user's posture, information 1020 related to an activity state of the user, and/or a plurality of pieces of detectable environment information 1030. The information 1010 related to the user's posture may include information indicating whether the user is in a standing state, a sitting state, or a lying state. The information 1020 related to the user's activity state may include information indicating whether the user is resting, sleeping, exercising, or moving. According to various embodiments, the plurality of pieces of environment information 1030 may include time information, weather information, country information (e.g., a country connected to a communication network) and/or location information. For example, the time information may include information such as time after waking up, time before bedtime, bedtime, time before meal or time after meal. For example, the bedtime is a time for the user to recover, and when the user sleeps enough according to the user's rhythm, the user's stress may be reduced and the user's blood pressure may be affected. In the case of mealtime, eating salty food may increase blood flow and increase the user's blood pressure. For example, as to the weather information and the location information, the user's blood pressure may be affected by the user's body rhythm. The weather information may affect the user's blood pressure according to a user's taste, and the location information may affect the user's blood pressure to correspond to a place the user frequently visits (e.g., home, office) and an unfamiliar place (e.g., a hospital).

According to various embodiments, the electronic device 101 may collect context information indicating the external environment by using at least one sensor included in the situation sensing module 320. The electronic device 101 may also consider the context information when automatically measuring the user's blood pressure using a biometric sensor (e.g., the biometric sensor 310 of FIG. 3). According to an embodiment, the electronic device 101 may map the context information and the user's biometric data and may store the mapped information in a memory (e.g., the memory 130 of FIG. 1).

According to an embodiment, when automatically measuring the user's blood pressure value during daily life, the electronic device 101 may also record the context information, and may provide the user with the amount of change in the blood pressure value according to a specific situation. The electronic device 101 may map the measured blood pressure value and the context information, and may the mapped information in the memory 130. According to an embodiment, the user may identify the blood pressure in a specific situation and may examine the user's health condition.

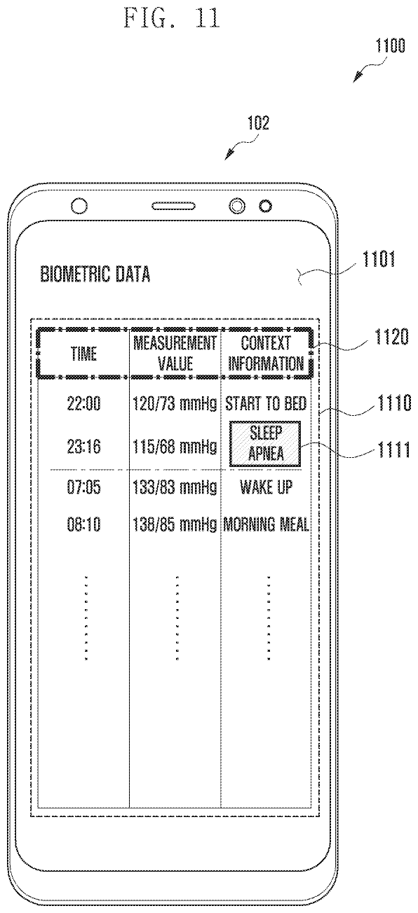

FIG. 11 is a diagram illustrating a list recorded based on biometric data measured by the electronic device 101 and context information of a user according to an embodiment of the disclosure.

Referring to FIG. 11 depicting diagram 1100, an electronic device (e.g., the electronic device 101 of FIG. 1 or a wearable device) may automatically measure a blood pressure value and may store context information at the time of measuring the blood pressure together. The wearable device 101 (e.g., the first electronic device) may perform wireless communication with a portable electronic device (e.g., the electronic device 102 or the second electronic device of FIG. 1), and may transmit the measured blood pressure value and the context information to the portable electronic device 102. According to an embodiment, the portable electronic device 102 may record the blood pressure and the context information transmitted from the wearable device 101 in a list, and may display a user interface (UI) 1110 including biometric data through a display device (e.g., the display device 1101 of the portable electronic device 102). For example, the user interface 1110 may display the measured blood pressure value based on a category 1120 including time information at which the blood pressure was measured, numerical information of the measured blood pressure, and/or context information. According to an embodiment, items included in the category 1120 are not limited and may be changed according to settings of a user and/or a developer.

According to various embodiments, the electronic device 101 may automatically detect a user's activity state instead of measuring the blood pressure after the user passively takes a mental and physical stable state, and may measure and record the user's blood pressure value when a specific condition is satisfied. In general, the blood pressure generally begins to rise just before waking up, continues to rise for about 1 to 2 hours, maintains during daily life, and falls at bedtime. A case in which the blood pressure measured at the hospital is in a normal range but the blood pressure measured at home is high may correspond to masked hypertension. A case in which the blood pressure measured at the hospital is high but the blood pressure measured at home is low may correspond to white-coat hypertension. In addition, a case in which a sharp increase in the blood pressure above about 135/85 mmHg in the morning may correspond to morning hypertension, and a case in which the blood pressure does not decrease below about 120/70 mmHg at bedtime may correspond to nocturnal hypertension. For example, morning hypertension is an important independent factor for stroke development and may be associated with cardiac hypertrophy and carotid hypertrophy, and nocturnal hypertension may be associated with sleep myocardial infarction and death from stroke. In general, when sleep apnea develops, the blood pressure may rise rapidly. According to an embodiment, the electronic device 101 may periodically measure the user's blood pressure, and may provide a notification message to the user when the blood pressure value is out of a predetermined normal blood pressure range. For example, the electronic device 101 may use a warning alarm and vibration to notify the user that a health problem has occurred. According to an embodiment, the electronic device 101 may highlight and display biometric data in which the health problem occurs based on the user interface 1110. For example, the electronic device 101 may highlight the occurrence information of sleep apnea 1111.

According to various embodiments, the wearable device 101 (e.g., the first electronic device) may automatically measure and record blood pressure during daily life, and may map the measured and recorded blood pressure together with context information to store the mapped information in the memory 130. The wearable device 101 may transmit the blood pressure and the context information to the portable electronic device 102 (e.g., the second electronic device). The portable electronic device 102 may generate the UI 1110 based on the transmitted blood pressure and context information, and may display the user interface 1110 through the display device 1101. According to an embodiment, the user may infer his or her health state and may prevent disease by considering the blood pressure automatically measured during daily life and the context information at the measurement time together.

FIG. 12 is a diagram illustrating a process of measuring blood pressure of a user by an electronic device according to an embodiment of the disclosure.

Referring to FIG. 12, in first graph 1201, an electronic device (e.g., the electronic device 101 of FIG. 1 or a wearable device) may use a biometric sensor (e.g., the biometric sensor 310 of FIG. 3) to acquire at least one PPG signal (e.g., a PPG signal divided based on PPG duration). The electronic device 101 may calculate an ensemble average based on the acquired at least one PPG signal. In second graph 1003, the electronic device 101 may overlap a plurality of PPG signals to calculate an ensemble average value 1210.

According to an embodiment, the electronic device 101 may calculate the PPG duration 1205 from the ensemble average value 1210, and may divide about 1 minute (e.g., 60 seconds) by the PPG duration 1205, thereby measuring a pulse rate of the user corresponding to the blood pressure. According to an embodiment, the electronic device 101 may measure the user's blood pressure based on the measured pulse rate.

According to an embodiment, the second graph 1203 may be a graph indicating a template of the PPG signal. For example, an absolute blood pressure value serving as the measurement reference may be configured to be about 120 for systolic blood pressure (SBP) and about 80 for diastolic blood pressure (DBP). According to an embodiment, the electronic device 101 may determine a relative difference value between the absolute blood pressure value and the blood pressure value measured by the PPG signal, and may measure the user's blood pressure based on the relative difference value.

According to an embodiment, the electronic device 101 may store a blood pressure algorithm for measuring blood pressure in a memory (e.g., the memory 130 of FIG. 3), and may measure the user's blood pressure using the blood pressure algorithm According to an embodiment, the electronic device 101 may reflect the biometric data extracted from the user and the reference biometric data stored in the memory 130 to the blood pressure algorithm, and may measure the user's blood pressure.

A blood pressure measurement method of the electronic device 101 according to various embodiments may include identifying whether a user is in an inactive state using a situation sensing module (e.g., the situation sensing module 320 of FIG. 3) including at least one sensor, identifying reference biometric data corresponding the inactive state when the user is in the inactive state, extracting biometric data of the user using a biometric sensor (e.g., the biometric sensor 310 of FIG. 3), and measuring blood pressure of the user based on the extracted biometric data and the reference biometric data.

In the method according to an embodiment, the identifying of whether the user is in the inactive state may include measuring a sensing value corresponding to the user's movement for a predetermined time by using an acceleration sensor and a gyro sensor included in the situation sensing module, and identifying that the user is in the inactive state when the sensing value corresponding to the movement does not exceed a threshold value.

The method according to an embodiment may further include identifying whether the user is in a sleeping state based on at least one the acceleration sensor, the gyro sensor, and the biometric sensor, and extracting the user's biometric data using the biometric sensor when the user is in the sleeping state.

The method according to an embodiment may further include identifying whether the user is sleeping in a sleep apnea state using a microphone included in the situation sensing module, and extracting the user's biometric data in response to the sleep apnea state.

The method according to an embodiment may further include acquiring context information indicating an external environment by using the situation sensing module, and mapping the measured blood pressure and the context information and storing the mapped information in the memory.

According to an embodiment, the context information may include at least one of location information, posture information, time information, surrounding people information, noise information, health-related information, and sleep phase information.

The method according to an embodiment may further include calculating first reference biometric data based on blood pressure data measured using a cuff blood pressure monitor in a calibration operation and a first weight corresponding to a first user posture, and calculating second reference biometric data based on the blood pressure data measured using the cuff blood pressure monitor and a second weight corresponding to a second user posture.

The method according to an embodiment may further include extracting the user's biometric data acquired using the biometric sensor in a blood pressure measurement operation, comparing the extracted user's biometric data with at least one of the first reference biometric data and the second reference biometric data to identify a relative difference value corresponding to the extracted user's biometric data, and measuring the user's blood pressure based on the identified difference value.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively,” as “coupled with,” “coupled to,” “connected with,” or “connected to” another element (e.g., a second element), it denotes that the element may be coupled with the other element directly (e.g., wiredly), wirelessly, or via a third element.

As used herein, the term “module” may include a unit implemented in hardware, software, or firmware, and may interchangeably be used with other terms, for example, “logic,” “logic block,” “part,” or “circuitry.” A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply denotes that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., Play Store™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An electronic device comprising:
a biometric sensor configured to detect biometric data;
a situation sensing circuit;
a memory; and
a processor operatively coupled to the biometric sensor, the situation sensing circuit, and the memory,
wherein the processor is configured to:
identify whether a user is in an inactive state using the situation sensing circuit,
identify reference biometric data corresponding to the inactive state in case that the user is in the inactive state,
extract biometric data of the user using the biometric sensor, and
measure a blood pressure of the user based on the extracted biometric data and the reference biometric data.

2. The electronic device of claim 1, wherein the processor is further configured to:
measure a sensing value corresponding to movement of the electronic device for a predetermined time using an acceleration sensor and a gyro sensor included in the situation sensing circuit; and
identify that the user is in the inactive state in case that the sensing value corresponding to the movement does not exceed a threshold value.

3. The electronic device of claim 2, wherein the processor is further configured to:
identify whether the user is in a sleeping state based on at least one of the acceleration sensor, the gyro sensor, or the biometric sensor; and
extract the biometric data of the user using the biometric sensor in case that the user is in the sleeping state.

4. The electronic device of claim 3, wherein the processor is further configured to:
identify whether the user is sleeping in a sleep apnea state using a microphone included in the situation sensing circuit; and
extract the biometric data of the user to correspond to the sleep apnea state.

5. The electronic device of claim 1, wherein the processor is further configured to:
identify whether the user is in a speaking state using a microphone included in the situation sensing circuit; and
identify that the user is in the inactive state in case that the user is not in the speaking state.

6. The electronic device of claim 1,
wherein the processor is further configured to:
acquire context information indicating an external environment using at least one sensor included in the situation sensing circuit,
map the measured blood pressure and the context information, and
store the mapped information in the memory.

7. The electronic device of claim 6,
wherein the context information comprises at least one of location information, posture information, time information, surrounding people information, noise information, health-related information, or sleep phase information.

8. The electronic device of claim 1, wherein the processor is further configured to measure the blood pressure based on the extracted biometric data and the reference biometric data using a blood pressure measurement algorithm stored in the memory.

9. The electronic device of claim 1, wherein the processor is further configured to:
calculate first reference biometric data based on blood pressure data measured using a cuff blood pressure monitor and a first weight corresponding to a first user posture in a calibration operation; and
calculate second reference biometric data based on the blood pressure data measured using the cuff blood pressure monitor and a second weight corresponding to a second user posture.

10. The electronic device of claim 9, wherein the processor is further configured to:
provide a guidance corresponding to at least one of the first user posture or the second user posture to the user in the calibration operation; and
calculate at least one of the first reference biometric data or the second reference biometric data based on the guidance.

11. The electronic device of claim 9, wherein the processor is further configured to:
extract the biometric data of the user acquired using the biometric sensor in a blood pressure measurement operation;
compare the extracted biometric data of the user with at least one of the first reference biometric data or the second reference biometric data to determine a relative difference value corresponding to the extracted biometric data of the user; and
measure the blood pressure of the user based on the determined difference value.

12. The electronic device of claim 1,
wherein the biometric sensor comprises a photoplethysmography (PPG)-based blood pressure monitor and comprises a light emitting device and a light receiving device, and
wherein the processor is further configured to extract, in case that at least a portion of the light emitted from the light emitting device is reflected on skin where blood vessels are located and the reflected portion of the light is received through the light receiving device, the user's biometric data based on the received light.

13. A blood pressure measurement method comprising:
identifying whether a user is in an inactive state using a situation sensing circuit comprising at least one sensor;
identifying reference biometric data corresponding the inactive state in case that the user is in the inactive state;
extracting biometric data of the user using a biometric sensor; and
measuring blood pressure of the user based on the extracted biometric data and the reference biometric data.

14. The blood pressure measurement method of claim 13, wherein the identifying of whether the user is in the inactive state comprises:
measuring a sensing value corresponding to a movement of the user for a predetermined time by using an acceleration sensor and a gyro sensor included in the situation sensing circuit; and
identifying that the user is in the inactive state in case that the sensing value corresponding to the movement does not exceed a threshold value.

15. The blood pressure measurement method of claim 14, further comprising:
identifying whether the user is in a sleeping state based on at least one the acceleration sensor, the gyro sensor, and the biometric sensor; and
extracting the user's biometric data using the biometric sensor in case that the user is in the sleeping state.

16. The blood pressure measurement method of claim 15, further comprising:
identifying whether the user is sleeping in a sleep apnea state using a microphone included in the situation sensing circuit; and
extracting the user's biometric data in response to the sleep apnea state.

17. The blood pressure measurement method of claim 13, further comprising:
acquiring context information indicating an external environment by using the situation sensing circuit;
mapping the measured blood pressure and the context information; and
storing the mapped information in a memory.

18. The blood pressure measurement method of claim 17,
wherein the context information comprises at least one of location information, posture information, time information, surrounding people information, noise information, health-related information, or sleep phase information.

19. The blood pressure measurement method of claim 13, further comprising:
calculating first reference biometric data based on blood pressure data measured using a cuff blood pressure monitor in a calibration operation and a first weight corresponding to a first user posture; and
calculating second reference biometric data based on the blood pressure data measured using the cuff blood pressure monitor and a second weight corresponding to a second user posture.

20. The blood pressure measurement method of claim 19, further comprising:
extracting the user's biometric data acquired using the biometric sensor in a blood pressure measurement operation;
comparing the extracted user's biometric data with at least one of the first reference biometric data or the second reference biometric data to identify a relative difference value corresponding to the extracted user's biometric data; and
measuring the user's blood pressure based on the identified difference value.
